SEQUENCE LISTING (3 sequences)

SEQ ID NO: 1 - protein, 21 aa, Homo sapiens
IDTKEGILQYCQEVYPELQIT

SEQ ID NO: 2 - protein, 16 aa, Homo sapiens
DAEFRHDSGYEVHHQK

SEQ ID NO: 3 - protein, 42 aa, Homo sapiens [sequence omitted]

(19) United States
(12) Patent Application Publication
(10) Pub. No.: US 2017/0143769 A1
(43) Pub. Date: May 25, 2017

(54) HUMAN UMBILICAL CORD BLOOD-DERIVED MONOCYTES FOR TREATMENT OF NEURODEGENERATIVE DISEASES AND DISORDERS

(71) Applicants: Jun Tan, Tampa, FL (US); Paul R. Sanberg, Spring Hill, FL (US)

(72) Inventors: Jun Tan, Tampa, FL (US); Paul R. Sanberg, Spring Hill, FL (US)

(73) Assignee: University of South Florida, Tampa, FL (US)

(21) Appl. No.: 15/360,023

(22) Filed: Nov. 23, 2016

Related U.S. Application Data
(60) Provisional application No. 62/258,649, filed on Nov. 23, 2015.

(51) Int. Cl. A61K 35/51; A61K 9/00

(52) U.S. Cl. CPC A61K 35/51; A61K 9/0019; A61K 2035/124

(57) ABSTRACT

Human umbilical cord blood-derived monocytes that markedly promote Aβ clearance through heterodimerization of sAPPα with Aβ and resultant sAPPα production for prevention or treatment of Alzheimer's disease and other neurodegenerative disorders (including stroke and TBI). It was discovered that multiple low-dose infusions of human umbilical cord blood cells (HUCBCs) ameliorate cognitive impairments and reduce Aβ-associated neuropathology in PSAPP transgenic mice, which markedly promotes amyloid precursor protein (APP) α-cleavage and resultant sAPPα production for pharmaceutical purposes, in particular for treating or slowing the progression of Alzheimer's disease.

### CROSS-REFERENCE TO RELATED APPLICATIONS

This nonprovisional application is claims priority to U.S. Provisional Patent Application No. 62/258,649, entitled “Human Umbilical Cord Blood-Derived Monocytes for Treatment of Neurodegenerative Diseases and Disorders”, filed Nov. 23, 2015 by the same inventors, the entirety of which is incorporated herein by reference.

### FEDERALLY SPONSORED RESEARCH OR DEVELOPMENT

This invention was made with Government support under Grant No. R01AG032432 awarded by the National Institutes of Health. The government has certain rights in the invention.

### BACKGROUND OF THE INVENTION

1. Field of the Invention

This invention relates, generally, to treatments for neurodegenerative diseases and disorders, such as Alzheimer's disease, stroke, and traumatic brain injuries. More specifically, it relates to human umbilical cord blood-derived monocytes for treatment of such neurodegenerative diseases and disorders.

2. Brief Description of the Prior Art

Alzheimer's disease (AD) is the fourth major cause of mortality in the elderly in the United States and the leading cause of dementia worldwide. Currently, 26 million people worldwide suffer from Alzheimer's disease (AD). Indeed it is predicted that the number of AD cases will quadruple and reach worldwide epidemic proportions by 2050. While pharmacological targets have been discovered, there are no true disease-modifying therapies.

β-amyloid plaques, resulting from defective removal or upregulated build up, are a central manifestation of AD (39). The role of Aβ in AD progression has already been established (19,34). The toxic function of this peptide in the brain is reported in many studies. However, the brain-specific receptor for this peptide has not yet been characterized. In many studies, it has been shown that scavenger receptors and LRP internalize Aβ. Yazawa and colleagues, for example, reported that formyl peptide receptor like-1, G protein coupled receptor usually found in monocytes and microglia and interact with Aβ species causing both internalization and inflammation (51). Generally, Aβ accumulation in the brain stimulates an influx of microglia, monocytes, and various pro-inflammatory cytokines adjacent to that area which causes localized inflammation and chemoattracts many more immune cells to that area.

It has been proposed that mechanisms involved in amyloid-β (Aβ) clearance might be key to providing insights into therapeutic interventions. The binding of Aβ to receptors that can mediate its clearance, has therefore been a major therapeutic focus (24,41,43,35,10). Aβ clearance via enzyme-mediated degradation, anti-Aβ autoantibodies, receptor-mediated Aβ transport across the BBB, or even therapeutic Aβ clearance has been encouraging, but not curative (4,27). It was shown that HUCBC-mononuclear cells (MNCs) could modulate inflammation and reduce Aβ pathology in combination with markedly rescuing cognitive deficits (9,36).

Additionally, there is a possibility that the amyloid precursor protein (APP) metabolite, soluble amyloid precursor protein α (sAPPα), may bind to Aβ and this may confer beneficial effects for the treatment of AD via increasing its clearance from the brain parenchyma (2,11).

HUCBCs administration has gained positive acceptance over the years in research and clinical settings as a source of hematopoietic stem cells to treat acquired and genetic diseases. HUCBCs has been used successfully to improve outcomes in many diseases especially in hematological malignancies (6), spinal cord injury (38), traumatic brain injury (29), stroke (7), cardiovascular disease (21,28) and preclinical models of AD (9,36). Brunstein et al. summarized a significant report on the use of allogenic umbilical cord blood cells in hematological malignancies especially in acute leukemia patients. The report suggested the use of cord blood cells under certain conditions, especially when a suitable sibling donor or a conventional donor are unavailable. However, the report data were taken from a single institution. Cord blood stem cells were used successfully in one study involving a spinal cord injured rat animal model, showing that infusion of human cord blood cells can reverse behavioral deficits even after five days of administration. Exogenous delivered cord blood cells were found around the injured area (38). Intravenous delivery of human cord blood cells after one day in a stroke animal model improved behavioral effects and stem cells were found around injured area. In this study the animal was immunocompromised with cyclosporine before administration of cells (46). Human umbilical cord blood progenitor cells were also used to treat acute myocardial infarction in a rat model. It was shown that exogenous cells could reduce infarction size significantly (21). Intravenous delivery of HUCBC-derived CD133⁺ cells improves functional recovery by preventing scar thinning and systolic dilation in a rat myocardium infarction model. (28). The current inventors have also used HUCBCs in AD mice models, showing that multiple low dose infusions of HUCBCs could improve cognitive function and reduce amyloid pathologies (9).

HUCBCs comprise a population of hematopoietic stem and progenitor cells. HUCBCs include about 95%-98% MNCs, which may contribute to the beneficial effects observed in AD. As part of their immune surveillance, monocytes phagocytose dead cells and other cellular debris (40). Therefore, monocytes may be fundamental to developing effective therapies for AD. It has been postulated that monocytes may be instrumental for enhancing Aβ clearance (31). It is known that resident microglia and perivascular macrophages are responsible for phagocytic clearance of Aβ from the brain parenchyma. A very early study in a rat model by Miyake and colleagues in 1984 showed that peripheral monocytes can enter into the brain after birth and can be detected for up to three weeks. They also found that those cells become morphologically changed in brain microenvironment and form amoeboid-like microglia (33). Moreover, peripheral monocytes have been reported to infiltrate the brain parenchyma in various disease conditions such as experimental allergic encephalomyelitis (15), trauma (3,26), neurological disease (49), and infection (13,25,30). Recent studies also show trafficking and infiltration of peripheral monocytes into the brain in psychological stress (49,47,48). It has been shown that peripheral inflammation and TNFα signaling also recruit monocytes into the brain from blood (12).

Although recent advances in cellular and molecular targets have yielded strategies for enhancing Aβ clearance and reversing AD-associated Aβ deposition—for example U.S. patent application Ser. No. 12/706,510; William V. Nikolic et al., Peripherally Administered Human Umbilical Cord Blood Cells Reduce Parenchymal and Vascular 3-Amyloid Deposits in Alzheimer Mice. Stem Cells and Development. June 2008, 17(3): 423-440. doi:10.1089/scd.2008.0018; Ende N et al., Human umbilical cord blood cells ameliorate Alzheimer's disease in transgenic mice, Journal of medicine (2001), 32, (3-4), 241-7; and Donna Darlington et al., Multiple Low-Dose Infusions of Human Umbilical Cord Blood Cells Improve Cognitive Impairments and Reduce Amyloid-β-Associated Neuropathology in Alzheimer Mice, Stem Cells and Development. Feb. 1, 2013, 22(3): 412-421. doi:10.1089/scd.2012.0345—a cure remains elusive.

Accordingly, what is needed is a more effective treatment for Aβ clearance and reversing neurological disorder-associated Aβ deposition. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for improved treatments for neurodegenerative diseases and disorders is now met by a new, useful, and nonobvious invention.

In an embodiment, the current invention is a method of preventing or treating a neurodegenerative disease or disorder (e.g., Alzheimer's disease, stroke, traumatic brain injury), comprising administering a therapeutically effective amount of human umbilical cord blood-derived monocytes. These monocytes may be pretreated with exogenous soluble amyloid precursor protein alpha and may be administered in multiple low-dose infusions.

In a separate embodiment, the current invention is a composition comprising sAPPα-treated aged monocytes formed by heterodimerization of sAPPα with amyloid beta. In yet another embodiment, the current invention is a method of preventing or treating a neurodegenerative disease or disorder (e.g., Alzheimer's disease, stroke, traumatic brain injury), comprising administering a therapeutically effective amount of this composition.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A depicts FACS analysis of surface expression markers in human umbilical cord blood monocytes, such as CD14⁺, total surface APP, and full-length APP, using anti-CD14⁺ antibodies by flow cytometry from HUCBC and HUCBC-derived monocytes.

FIG. 1B depicts FACS analysis of surface expression markers in human umbilical cord blood monocytes, such as CD14⁺, total surface APP, and full-length APP, using anti-Aβ-N-terminal (6E10) antibodies by flow cytometry from HUCBC and HUCBC-derived monocytes.

FIG. 1C depicts FACS analysis of surface expression markers in human umbilical cord blood monocytes, such as CD14⁺, total surface APP, and full-length APP, using anti-APP-C-terminal (pAb751/770) antibodies by flow cytometry from HUCBC and HUCBC-derived monocytes.

FIG. 1D depicts cell lysates obtained from HUCBC and HUCBC-derived monocytes subjected to WB analysis. The percentage of CD14, 6E10, and pAb751/770 APP positive cells in each quadrant is indicated. Overall, the purity of HUCBC-derived monocytes was found to be almost 95%.

FIG. 2A is a schematic illustration of HUCBC treatment schedule and testing for cognitive and motor function in PSAPP and WT mice. Both PSAPP and age-matched WT control mice were tested after short term systemic administration of monocytes.

FIG. 2B shows that the cognitive function was presented as numbers of entry-arm errors before finding the platform. WCB- and CB-M-treated PSAPP mice showed better behavioral performance than PBS treated group. In addition, CB-M-treated PSAPP mice showed improve behavioral performance than WCB-treated group. In contrast, MD-CB-treated group showed no cognitive improvement. WT littermates also showed similar tendency at each treatment condition. All data are presented as mean±SEM (*P<0.05; **P<0.01), and analyzed by one-way analysis of variance (ANOVA) with LSD post-hoc test.

FIG. 2C shows that motor function was also assessed using rotarod test after treatment. WCB-treated PSAPP mice showed enhance motor activity, reflected as enhanced latency to fall, but not statistically significant compared to PBS-treated group. CB-M-treated PSAPP mice showed significantly improved motor activity compared to MD-CB-treated mice on day 1. Similarly, CB-M-treated WT control mice showed significant improvement in motor activity compared to MD-CB-treated group on both days 1 and 2. All data are presented as mean±SEM (*P<0.05; **P<0.01), and analyzed by one-way analysis of variance (ANOVA) with one-way ANOVA with Bonferroni post-hoc test. Overall, FIGS. 2A-2C show that short term (two-month) administration of HUCBC-derived monocytes improves learning, memory and motor function.

FIG. 3A is a schematic diagram of HUCBC treatment schedule and behavioral testing in PSAPP and WT mice. Both PSAPP and aged-match WT control mice were cognitively tested after long term monocytes administration.

FIG. 3B shows that hippocampal-dependent learning and memory function was assessed by RAWM test after treatment completion. The cognitive activity was presented as numbers of entry-arm errors before finding the platform. Consistent with the two-month treatment data, WCB- and CB-M-treated PSAPP mice showed improved cognitive function compared with PBS-treated PSAPP mice. WT littermates also showed similar effects with each treatment paradigm. MD-CB-treatment did not show behavioral improvement, and even exacerbated the cognitive function of WT mice. All data are shown as the mean±SEM (*P<0.05; **P<0.01) and analyzed by one-way analysis of variance (ANOVA) with LSD post-hoc test.

FIG. 3C shows that motor activity was assessed using rotarod test after monocytes administration. WCB- or CB-M-treated PSAPP mice significantly improve the motor activity, assessed as latency to fall, compared to PBS-treated group, while MD-CB treatment progressively impaired the motor function of WT control group. All data are shown as the mean±SEM (*P<0.05; **P<0.01) and analyzed by one-way analysis of variance (ANOVA) with one-way ANOVA with Bonferroni post-hoc test. Overall, FIGS. 3A-3C show that long term (four-month) administration of HUCBC-derived monocyte improves learning, memory and motor function.

FIG. 4A shows that HUCBC (WCB) and HUCBC-derived monocytes (CB-M) significantly reduce β-amyloid plaques in retrosplenial cortex (RSC), entorhinal cortex (EC), and hippocampus (H) of PSAPP mice, as determined by immunohistochemical staining with anti-Aβ₁₇₋₂₄ antibody (4G8).

FIG. 4B shows that the percentage quantification of immunoreactive areas in area of interest (H region) by image analysis for each treatment group using 4G8 antibody (n=4).

FIG. 4C shows that detergent-soluble Aβ₁₋₄₀ and Aβ₁₋₄₂ levels in brain homogenates of PSAPP mice were analyzed by Aβ ELISA. Data are represented as mean±SD of Aβ_{1-40,42} (ng/mg of total protein). A t-test for independent samples reveals significant differences between HUCBC or HUCBC-derived monocyte (CB-M) treatment and PBS control groups (**P<0.01), but no significant difference between whole HUCBC (WCB) and CB-M treatment groups (P>0.05). Overall, FIGS. 4A-4C show that HUCBC-derived monocytes reduce Aβ pathology.

FIG. 5A depicts a quantitative measurement of Aβ₁₋₄₂ internalization by cord and aged blood monocytes. Extracellular (upper panel) and cell-associated (lower panel) FITC-Aβ₁₋₄₂ was measured from supernatants and lysates respectively using fluorimeter. HUCBC-derived monocytes (CB-M) shows lower level of extracellular Aβ₁₋₄₂ (top left panel) and as expected higher level of cell-associated Aβ₁₋₄₂ than aged BC-M (bottom left panel). Internalization of Aβ₁₋₄₂ by aged human blood cell-derived monocytes (BC-M) was enhanced by sAPPα treatment (100 ng/ml). Monocyte scavenger receptor class A (SR-A) appears to be the cell surface receptor that account for the internalization Aβ₁₋₄₂ SR-A ligand decreased Aβ₁₋₄₂ internalization during sAPPα treatment. Data are represented as relative mean fluorescence (mean±SD) for each sample at 37° C. divided by mean fluorescence at 4° C. (n=4 for each condition) (*P<0.05, **P<0.01, *** P<0.001).

FIG. 5B depicts in vitro confocal images of Aβ phagocytosis by cord and aged blood derived monocytes. Monocytes were obtained, characterized by flow cytometry, incubated with purified FITC-Aβ₁₋₄₂ peptide (1 μM) for 60 minutes, and then assessed by confocal microscopy. Primary monocytes were treated, fixed and imaged using confocal microscopy equipped with Normarski optics. Blue fluorescence represents monocytes and green fluorescence represents FITC-labeled Aβ₁₋₄₂. Merged image shows significant internalization of Aβ₁₋₄₂ within the cytoplasm of CB-M as shown by most of the green fluorescence localized in the cytoplasmic compartment (left upper panel) while aged BC-M shows significant reduction of Aβ₁₋₄₂ internalization (left lower panel). Aged BC-M treated with sAPPα shows significantly increased Aβ₁₋₄₂ internalization in the cytoplasm (right upper panel), while SR-A ligand decreased Aβ₁₋₄₂ internalization inside the cytoplasm during sAPPα treatment (right middle panel). Unlabeled (naked) Aβ₁₋₄₂ competitively reduced internalization of FITC-Aβ₄₂ by CB-M (left middle panel).

FIG. 6A shows conditioned media of CHO-APPwt and CHO-APPswe cells expressing APP, Aβs and sAPPα immunoprecipitated with anti-Aβ₁₇₋₂₇ antibody (4G8), followed by WB detection with anti-Aβ₁₋₁₆ monoclonal antibody (6E10) (left panel) or an anti-APP N-terminal antibody (22C11) (right panel). WB with 6E10 shows both sAPPα and Aβ bands (left panel), but WB with 22C11 only shows sAPPα band (right panel).

FIG. 6B depicts further confirmation of heterodimerization by immunoprecipitating with anti-APP N-terminal antibody (22C11) and WB detection with 6E10, revealing both sAPPα and Aβ bands. Overall, FIGS. 6A-6B prove sAPPα and Aβ heterodimerization at monocyte's cell surface via immunoprecipitation of sAPPα and Aβs in cell culture.

FIG. 7A is a diagram showing the sequence of truncated sAPPα (top) and Aβ₄₂ (bottom). Note that Ahx (aminohexanoic acid), a common hydrophobic spacer.

FIG. 7B depicts analysis by WB with anti-Aβ₁₋₁₆ antibody (82E1) after truncated sAPPα was incubated with synthesized Aβ₁₋₄₂ and immunoprecipitated with anti-Aβ₁₇₋₂₇ antibody (4G8). For negative control, conditioned media from CHO/APPswe cells underwent the same treatment in this experiment. 82E1 picked up both Aβ₁₋₄₂ (˜4 kDa, upper band) and truncated sAPPα (˜4 kDa, lower band) after incubating truncated sAPPα with synthesized Aβ₄₂ (lanes 1 and 2) but only Aβ₁₋₄₂ band showed up in control (lane 3).

FIG. 7C depicts analysis by WB with an anti-APP N-terminal monoclonal antibody (22C11) after truncated sAPPα was incubated with synthesized Aβ₁₋₄₂ and immunoprecipitated with anti-Aβ₁₇₋₂₇ antibody (4G8). 22C11 only picked up truncated sAPPα (˜4 kDa) (lanes 1 and 2) after truncated sAPPα was incubated with synthesized Aβ₄₂ and as expected only sAPPα (˜100 kDa) (lane 3) in CHO/APPswe conditioned media. Overall, FIGS. 7A-7C prove that APP₆₇₂₋₆₈₇ (Aβ₁₋₁₆) region is responsible for sAPPα/Aβ heterodimerization.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a”, “an”, and “the” include plural referents unless the content clearly dictates otherwise. As used in this specification and the appended claims, the term “or” is generally employed in its sense including “and/or” unless the context clearly dictates otherwise.

As used herein, “about” means approximately or nearly and in the context of a numerical value or range set forth means ±15% of the numerical. In an embodiment, the term “about” can include traditional rounding according to significant figures of the numerical value. In addition, the phrase “about ‘x’ to ‘y’” includes “about ‘x’ to about ‘y’”.

As used herein, “treat”, “treatment”, “treating”, and the like refer to acting upon a condition (e.g., neurodegenerative disease/disorder or symptom thereof) with an agent (e.g., HUCBC-derived monocytes, sAPPα-treated aged monocytes) to affect the condition by improving or altering it. The improvement or alteration may include an improvement in symptoms or an alteration in the physiologic pathways associated with the condition. The aforementioned terms cover one or more treatments of a condition in a patient (e.g., a mammal, typically a human or non-human animal of veterinary interest), and includes: (a) reducing the risk of occurrence of the condition in a subject determined to be predisposed to the condition but not yet diagnosed, (b) impeding the development of the condition, and/or (c) relieving the condition, e.g., causing regression of the condition and/or relieving one or more condition symptoms.

As used herein, the terms “prophylactically treat” or “prophylactically treating” refers completely or partially preventing (e.g., about 50% or more, about 60% or more, about 70% or more, about 80% or more, about 90% or more, about 95% or more, or about 99% or more) a condition or symptom thereof and/or may be therapeutic in terms of a partial or complete cure or alleviation for a condition and/or adverse effect attributable to the condition.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one or more such excipients, diluents, carriers, and adjuvants.

The term “therapeutically effective amount” as used herein describes concentrations or amounts of components such as agents which are effective for producing an intended result. Compositions according to the present invention may be used to effect a favorable change in the neurodegenerative disease/disorder or symptom thereof, whether that change is an improvement, relieving to some extent one or more of the symptoms of the condition being treated, and/or that amount that will prevent, to some extent, one or more of the symptoms of the condition that the host being treated has or is at risk of developing, or a complete cure of the disease or condition treated.

The term “administration” or “administering” is used throughout the specification to describe the process by which a composition comprising HUCBC-derived monocytes or sAPPα-treated aged monocytes as active agent, are delivered to a patient or individual for therapeutic purposes. The composition of the subject invention and methodology in use thereof can be administered a number of ways including, but not limited to, parenteral (such term referring to intravenous and intra-arterial as well as other appropriate parenteral routes), subcutaneous, peritoneal, inhalation, vaginal, rectal, nasal, or instillation into body compartments.

Administration will often depend upon the amount of compound administered, the number of doses, and duration of treatment. In an embodiment, multiple doses of the agent are administered. The frequency of administration of the agent can vary depending on any of a variety of factors, such as stage of the neurodegenerative disease/disorder, and the like. The duration of administration of the agent, e.g., the period of time over which the agent is administered, can vary, depending on any of a variety of factors, including patient response, etc.

The amount of the agent contacted (e.g., administered) can vary according to factors such as the degree of susceptibility of the individual, the age, sex, and weight of the individual, idiosyncratic responses of the individual, the dosimetry, and the like. Detectably effective amounts of the agent of the present disclosure can also vary according to instrument and film-related factors. Optimization of such factors is well within the level of skill in the art, unless otherwise noted.

As used herein, the term “subject,” “patient,” or “organism” includes humans and mammals (e.g., mice, rats, pigs, cats, dogs, and horses). Typical hosts to which an agent(s) of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range.

Discussed herein are human umbilical cord blood-derived monocytes that markedly promote Aβ clearance through heterodimerization of sAPPα with Aβ and resultant sAPPα production for prevention or treatment of Alzheimer's disease and other neurodegenerative disorders (including stroke and TBI). It was discovered that multiple low-dose infusions of human umbilical cord blood cells (HUCBCs) ameliorate cognitive impairments and reduce Aβ-associated neuropathology in PSAPP transgenic mice, which markedly promotes amyloid precursor protein (APP) α-cleavage and resultant sAPPα production for pharmaceutical purposes, in particular for treating or slowing the progression of Alzheimer's disease.

Study

The current inventors have reported that human umbilical cord blood cells (HUCBCs) modulated inflammation, diminished Aβ pathology, and reduced behavioral deficits in PSAPP transgenic mice (9). Based on the prior art and the current inventors' previous study (9,36), the present study sought to modify the current inventors' previous HUCBCs study in such a way that only the CD14⁺ monocyte fraction (HUCBC-derived monocytes), isolated from total HUCBCs, would be infused peripherally over short and long term periods of time in PSAPP and WT mice.

The present study attempts to determine which mononuclear cell (MNC) population was conferring these effects. Accumulated AD research has suggested that monocytes among MNCs are contributory to promoting Aβ clearance (9,31,32,20). Therefore, it was examined whether monocytes purified from HUCBCs would have beneficial outcomes on the reduction of AD-like pathology and rescue of cognitive impairments in PSAPP transgenic AD mice. PSAPP mice and their wild-type littermates were treated monthly with a peripheral HUCBC-derived monocyte infusion over a period of 2 and 4 months, followed by behavioral evaluations as well as biochemical and histological analyses. The present study extends on the HUCBCs AD therapy to: (1) examine whether monocytes are responsible for the beneficial effects against AD observed in HUCBCs therapeutics, and (2) determine the mechanism by which these salutary effects occur.

The current inventors discovered that multiple low-dose infusions of human umbilical cord blood cells (HUCBCs) ameliorate cognitive impairments and reduce Aβ-associated neuropathology in PSAPP transgenic mice. In the present study, it was examined whether monocytes, as important components of HUCBCs, would have beneficial outcomes on the reduction of AD-like pathology and associated cognitive impairments in PSAPP transgenic AD model mice. PSAPP mice and their wild-type littermates were treated monthly with an infusion of peripheral human umbilical cord blood cell (HUCBC-derived monocytes over a period of 2 and 4 months, followed by behavioral evaluations, biochemical, and histological analyses. The principal findings of the present study confirmed that monocytes derived from HUCBCs (CB-M) play a central role in HUCBC-mediated cognition-enhancing and Aβ-pathology ameliorating activities.

Importantly, it was found that compared with CB-M, aged monocytes showed an ineffective phagocytosis of Aβ, while exogenous soluble amyloid precursor protein alpha (sAPPα) could reverse this deficiency. Pretreating monocytes with sAPPα upregulates Aβ internalization. Further studies suggested that sAPPα could form a heterodimer with Aρs, with the APP672-688 (Aβ1-16) region being responsible for this effect. This in turn promoted binding of these heterodimers to monocyte scavenger receptors and thus promoted enhanced Aβ clearance. In summary, the current study suggests that peripheral monocytes contribute to Aβ clearance through heterodimerization of sAPPα with Aβ. Further, declined or impaired sAPPα production, or reduced heterodimerization with Aβ, would cause a deficiency in Aβ clearance and thus accelerate the pathogenesis of AD.

### A. MATERIALS AND METHODS

All experiments were performed in accordance with the guidelines of the National Institutes of Health, and all animal studies were approved by the University of South Florida (USF) Institutional Animal Care and Use Committee. Animals were humanely cared for during all experiments, and all efforts were made to minimize animal suffering. Animals were anesthetized with isoflurane (50 mg/kg) and euthanized by transcardial perfusion with ice-cold physiological saline containing heparin (10 units/mL). In this study, use of human cord blood cells was involved. Ninety-five to 98% mononuclear cells from HUCBCs were provided by SANERON CCEL Therapeutics Inc. (Tampa, Fla.). SANERON used de-identified HUCBCs donations from certified commercial sources.

i. Animals

PSAPP (APPswe/PS1DE9) mice and their wild-type (WT) littermates were obtained from the Jackson Laboratory (Bar Harbor, Me.). PSAPP mice overproduce human Aβ₁₋₄₀ and Aβ₁₋₄₂ peptides (APP Swedish mutation with a PS1 deletion in exon 9) and from about 4 months develop progressive cerebral Aβ deposits with learning and memory impairment, albeit without neuronal loss (9,5,17,1,23). All mice were characterized by PCR genotyping for mutant human APP and presenilin 1 (PS1) transgenes. Thus, all mice used in this study are genetically comparable. Both PSAPP mice and their WT littermates were maintained on a 12 hour-light/12 hour-dark cycle at ambient temperature and humidity. All cohorts were housed in the animal facility at the USF, Morsani College of Medicine (Johnnie B. Byrd Sr. Alzheimer's Center and Research Institute, Tampa, Fla.). Animals were fed standard rodent chow and water ad libitum. For the cord blood-derived monocyte experiments, 5.5-month old PSAPP mice and WT littermates were used. All cohorts were female and this served to eliminate the possibility of gender discrepancies among groups. All animals in these experiments were observed in a blinded, randomized approach.

ii. HUCBCs Preparation

HUCBCs, comprised of 95-98% mononuclear cells, were obtained using a proprietary density gradient solution (DSS-001) developed by SANERON and GE HEALTHCARE. Our purified HUCBCs were cryopreserved and stored in liquid nitrogen at −210° C. HUCBCs were thawed prior to transplantation, at 37° C. for 4 minutes, washed in 0.05 M phosphate-buffered saline (PBS), assessed for cell quantification and viability (CELLDYN, VI-CELL, Indianapolis Ind.), and suspended in PBS to achieve a cell concentration of 2×10⁵ cells per 100 μL for each infusion.

iii. HUCBC-Derived Monocyte Acquisition

The HUCBCs suspension was centrifuged and re-suspended in monocyte buffer, containing 0.5% bovine serum albumin (BSA), and 2 mM EDTA in PBS. Monocytes were separated via positive selection using the MACS Miltenyi biotec CD14⁺ MicroBeads human kit (MILTENYI BIOTEC Inc., San Diego, Calif.) (44). CD14⁺ MicroBeads were added to the cell suspension and incubated for 15 minutes at 4° C. Cells were washed, re-suspended in PBS and run through a column in a magnetic separator. Cells were allowed to flow into a collection flask and this comprised the monocyte-deficient cord blood (MD-CB) infused in the cohorts. The column was removed and flushed with buffer to obtain an enriched population of cord blood derived monocytes (CB-M). Acquired monocyte populations were characterized using flow cytometry. Cell counts and viability were then carried out as described. Cells were centrifuged, re-suspended in PBS twice and aliquots of 2×10⁵ cells per 100 μL concentration per mouse were made.

iv. Flow Cytometry Analysis of HUCBC-Derived Monocytes

HUCBC-derived monocytes were stained for identification of the subset monocyte population expressing CD14⁺ using an anti-CD14⁺ monoclonal antibody (SIGMA-ALDRICH, St. Louis, Mo.), and FACS analysis by ACCURI C6 Flow Cytometer (Rochester, Minn.). Only monocyte populations composed of ≧˜93% CD14⁺ cells were used in the study. In addition, further flow cytometry analysis of HUCBC-derived monocytes for membrane surface associated APP and full-length APP was conducted using an anti-Aβ N-terminal monoclonal antibody (6E10, 1:2,000; COVANCE RESEARCH PRODUCTS, Emeryville, Calif.) and an anti-APP C-terminal polyclonal antibody (pAb751/770, 1:1,000; CALBIOCHEM, Billerica, Mass.) according to manufacturer specifications.

v. Western Blot Analysis for APP Levels

Cell lysates from cultured HUCBCs and HUCBC-monocytes were assayed by Western blot (WB) analysis. In brief, cells were cultured then lysed in ice-cold lysis buffer (1 mM Na₃VO₄, 150 mM NaCl, 1 mM EDTA, 2.5 mM sodium pyrophosphate, 20 mM Tris pH 7.5, 1 mM EGTA, 1% v/v Triton X-100, 1 mM PMSF, 1 mM β-glycerolphosphate, 1 μg/mL leupeptin). Inherent proteins were separated using 10% gel, transferred to 0.2-μm nitrocellulose membranes (BIO-RAD, Hercules, Calif.) and visualized using standard immunoblotting protocol. All antibodies were diluted in 0.05 M Tris-buffered saline (TBS) containing 5% (w/v) nonfat dry milk. Membranes were immunoblotted with appropriate primary antibody and then probed using an anti-mouse IgG (1:2,000; CELL SIGNALING TECHNOLOGY, Danvers, Mass.) or an anti-rabbit IgG (1:10,000; THERMO FISHER SCIENTIFIC, Waltham, Mass.) secondary antibody conjugated with horseradish peroxidase. Proteins were detected with Super Signal West Femto Maximum Sensitivity Substrate (THERMO FISHER SCIENTIFIC, Waltham, Mass.) and BIOMAX-MR Film (THERMO FISHER SCIENTIFIC, Waltham, Mass.). Primary antibodies include an anti-APP N-terminal monoclonal antibody (22C11, 1:2,000, EMD Millipore, Temecula, Calif.), an anti-APP C-terminal polyclonal antibody (pAb751/770, 1:1,000) to evaluate APP expression, an anti-Aβ₁₋₁₆ monoclonal antibody (6E10, 1:2,000), and an anti-β-actin monoclonal antibody (1:4,000; SIGMA-ALDRICH).

vi. HUCBC-Derived Monocyte Infusion

Briefly, 23 PSAPP mice and WT littermates were randomly assigned into the following four treatment groups: whole HUCBCs (WCB, n=6), HUCBC-derived monocytes (CB-M, n=6), monocyte-deficient HUCBC (MD-CB, n=6), or PBS (n=5). With respect to the number of PSAPP and WT mice used in the study, each group, with the exception of the PBS treated cohorts, comprised n=3 animals per strain. The PBS treated group contained WT (n=2) and PSAPP (n=3). The right tail vein of PSAPP mice or their WT littermates was identified and vasodilated using warm water and then 2×10⁵ cells/100 μL WCB, CB-M, MD-CB or 100 μL PBS per mouse was delivered via right tail vein injection. The injection was performed four times over two months and six times over four months. HUCBCs were infused at the end of weeks 1, 3, 5 and 7 for the 2-month treatment and at the end of weeks 1, 3, 5, 7, 9, 11, 13 and 15 for the 6-month treatment. These agents were administered intravenously since it is a readily available approach and shown previously by the current inventors to be effective in reducing AD pathology in mice (9). However, other suitable methods of administration are contemplated herein as well.

vii. Behavioral Tests

Motor and cognitive evaluations were conducted at the end of the 2-month treatment (7.5 months of age) or at the end of the 4-month treatment (9.5 months of age) using the rotarod test for motor activity, as well as the radial arm water maze (RAWM) test and the visible platform in an open pool test for cognitive ability.

Rotarod test. For two consecutive days, mice underwent rotarod test. Mice were positioned on the rod (diameter 3.6 cm) of the equipment (ROTAROD 7650 accelerating model UGO BASILE, Biological Research Apparatus, Varese, Italy). The rod was set at 4.0 rpm and mice were placed 5 at a time on the rod. Trial time was five minutes and the rod steadily accelerated from 4.0 rpm up to 40.0 rpm. Mice were evaluated by the time they were able to retain their balance position on the rod.

Radial arm water maze test. All mice received 2 days of 15 swims or trials per day. Each swim culminated either when a visible or a submerged underwater goal was located or after 1 minute had elapsed. Briefly the mouse was dropped into a random start arm (predetermined on a score sheet) and allowed to swim until it located and climbed onto the platform (goal) over a period of 1 minute. Errors were recorded as any entry into an incorrect arm or failure to enter any arm for the initial 15 seconds of the trial. Results were analyzed as number of errors made.

Visible platform in an open pool swim test. For verification of whether the animals possessed the skills sufficient to complete the water maze task, the visible platform was used in an open pool swim test. In brief, it was performed in the same pool as the radial arm water maze test; however, the triangular wedges were removed and the pool was left open with a visible platform in an imagined quadrant. Latency to find and ascend the platform was measured (60 seconds maximum).

viii. Tissue Preparation

Subsequent to neurocognitive evaluations, cohorts were anesthetized with isoflurane and euthanized at either 7.5 or 9.5 months of age. Hind limbs (for bone marrow) and 500 peripheral blood were initially collected and the mice were then perfused transcardially with an ice-cold physiological saline. Brains were rapidly isolated and the left hemispheres were frozen immediately in liquid nitrogen and stored at −80° C. For molecular analysis, the left hemispheres were sonicated in RIPA buffer (Cell Signaling Technology) and centrifuged at 14,000 rpm for 1 hour at 4° C. Supernatant was transferred to a new tube for soluble Aβ analysis and the pellet was used for insoluble Aβ extraction as described previously (42). The right hemispheres were placed in 4% paraformaldehyde in PBS at 4° C. overnight, and then transferred to a graded series of sucrose solutions (10%, 20%, and 30%, each at 4° C. overnight) for cryostat sectioning. Sequential 25-μm coronal sections were cut and free-floating sections were then stored at 4° C. in 24-well plates containing PBS with 100 mM sodium azide.

ix. Immunohistochemical Analysis

Sections were immunohistochemically stained using an anti-Aβ₁₇₋₂₆ monoclonal antibody (4G8, 1:200; COVANCE RESEARCH PRODUCTS) in conjunction with the VECTASTAIN ABC Elite kit (VECTOR LABORATORIES, Burlingame, Calif.) coupled with diaminobenzidine substrate. For all the staining, a set of sections without adding primary antibody was used as negative staining control. Aβ burden (Aβ immunoreactive area) was determined by quantitative image analysis of Aβ plaques burden in the retrosplenial cortex (RSC), entorhinal cortex (EC), and hippocampus (H) brain regions of PSAPP mice and their WT littermates for each of the groups treated. Images of five 25-μm sections (150-μm apart) through hippocampus and neocortex (RSC, EC brain regions) were captured and a threshold optical density was obtained that discriminated staining from background. Quantification of 4G8 positive Aβ burden is reported as a percentage of immunolabeled area captured (positive pixels divided by total pixels captured). Quantitative image analysis was performed by a single examiner blinded to sample identities. Data are represented as mean±SD (n=4 females).

x. Enzyme-Linked Immunosorbent Assay

The enzyme-linked immunosorbent assay (ELISA) was performed according to the current inventors' previous methods (37). Soluble Aβ_{1-40/42} levels in brain homogenates were analyzed by ELISA using Aβ_{1-40/42} ELISA kits (INVITROGEN, Grand Island, N.Y.) in accordance with manufacturer's instructions. Data are represented as mean±SD of Aβ_{1-40/42} (ng/mg of total protein).

xi. Monocyte Phagocytosis Assay

Monocytes were acquired as described earlier from both HUCBCs and aged human blood cells (from senior adults over 70 years old). These cells were further characterized for cell surface CD14⁺ and APP biomarkers using flow cytometry analysis. Phagocytosis of Aβ was conducted as previously described (52). Briefly, primary HUCBC-derived monocytes (CB-M) and aged human blood cell-derived monocytes (aged BC-M) were incubated with 1 μM FITC-Aβ₁₋₄₂ for 1 hour in the absence or presence of sAPPα (100 ng/ml), scavenger receptor class A ligand (SR-A) or unlabeled Aβ (naked Ab₄₂). Both extracellular and cell-associated FITC-Aβ₁₋₄₂ from cellular supernatants and lysates were quantified using fluorometric analysis to determine mean fluorescence value for each sample as previously described (53). For each condition, relative fold change values were calculated as: mean fluorescence value for each sample at 37° C./mean fluorescence value for each sample at 4° C. In all of the conditions, an additional control without cells was carried out to account for nonspecific adherence of Aβ to the plastic surface of culture plates and the mean values were normalized to these controls. Monocytes were then fixed and imaged in independent channels using a confocal microscope equipped with Normarski optics.

xii. Cell Culture and Immunoprecipitation

Chinese Hamster ovary (CHO) cells overexpressing either WT human APP (CHO/APPwt) or Swedish mutant APP (APPswe) were donated by Dr. Stefanie Hahn and Dr. Sascha Weggen (University of Heinrich Heine, Düsseldorf, Germany). These cells were cultured for 3 hours and then the conditioned media were collected, immunoprecipitated using an anti-Aβ₁₇₋₂₆ monoclonal antibody (4G8), and then analyzed by WB using 22C11 or 6E10. Alternatively, the conditioned media were immunoprecipitated with 22C11 and then analyzed by WB using 6E10. For the parallel heterodimerization study, truncated sAPPα peptide was incubated with synthesized Aβ₁₋₄₂, immunoprecipated with 4G8 and analyzed by WB using 82E1 or 22C11. In addition, CHO/APPswe-derived conditioned media were collected, immunoprecipitated with 4G8 and then analyzed by WB using 82E1) or 22C11).

xiii. Statistical Analysis

All data were presented as mean±SEM or mean±SD and normally distributed. For the RAWM test, the one-way analysis of variance (ANOVA) followed by post-hoc LSD test was performed to compare differences between groups. For the rotarod test, data were analyzed by ANOVA followed by post-hoc Bonferroni test. For Aβ burden as well as both soluble and insoluble Aβ_{1-40/42}, a t-test for independent samples followed by a post hoc Bonferroni was used to determine the significant difference between each MD-CB-, WCB-, CB-M-, and PBS-treated group. A P value of <0.05 was considered significant. All analyses were performed using the Statistical Package for the Social Sciences (SPSS), release 18.0 (IBM, Armonk, N.Y.).

V. Results

i. Characterization of Purified Human Umbilical Cord Blood-Derived Monocytes

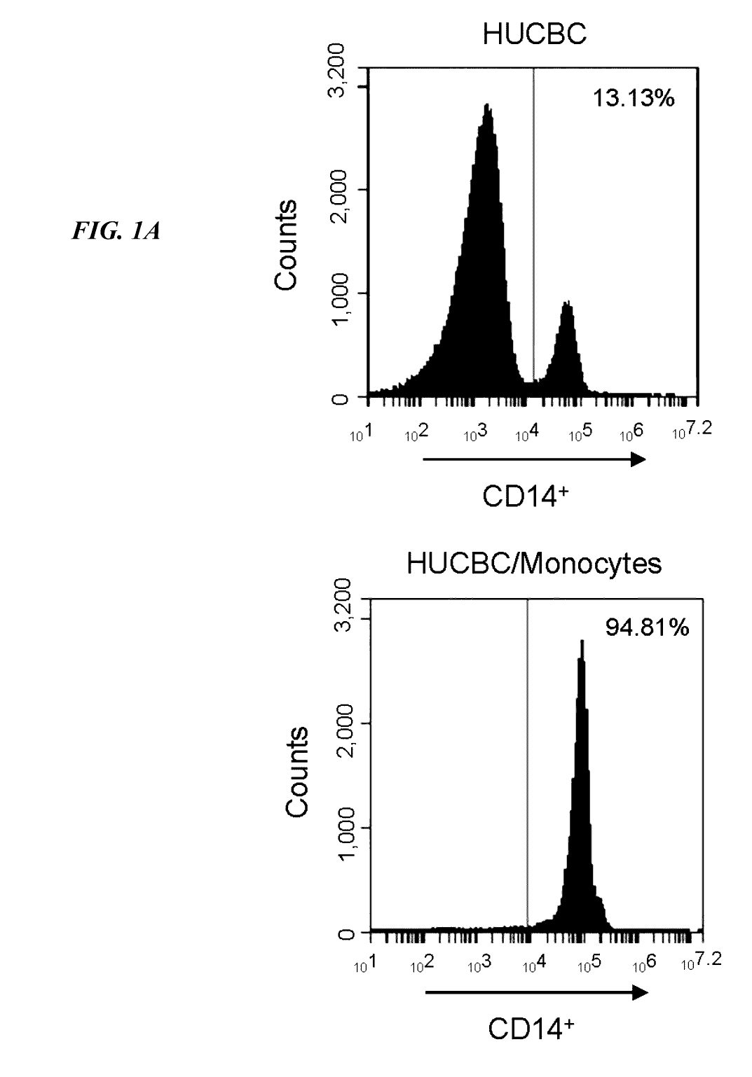

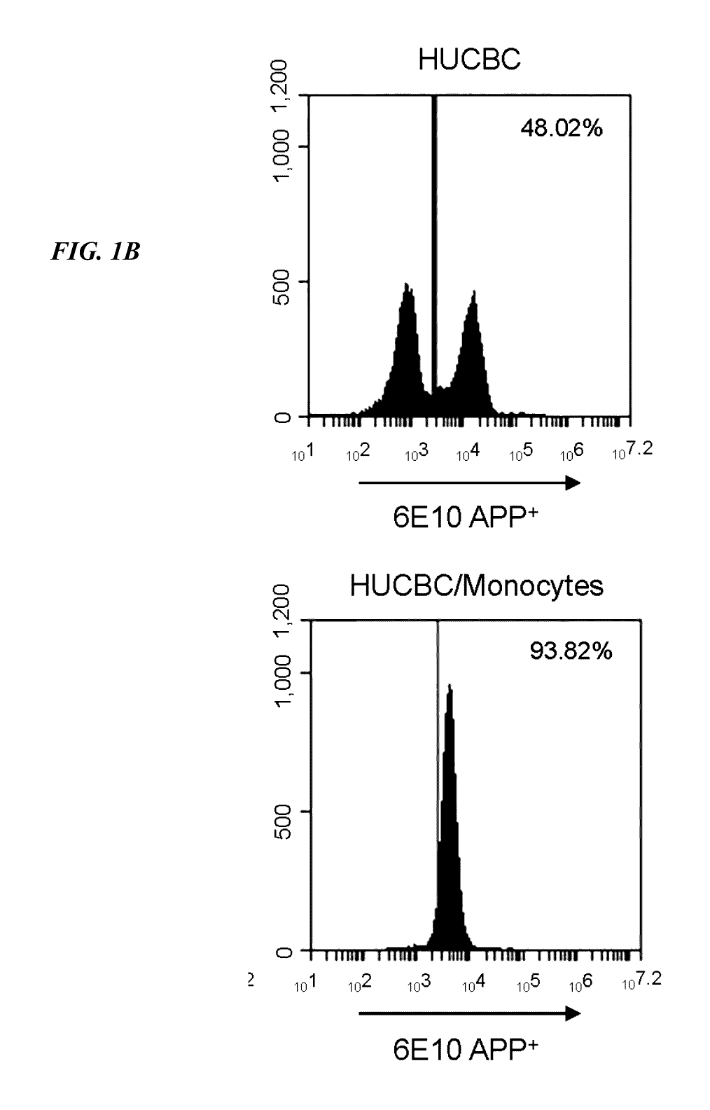

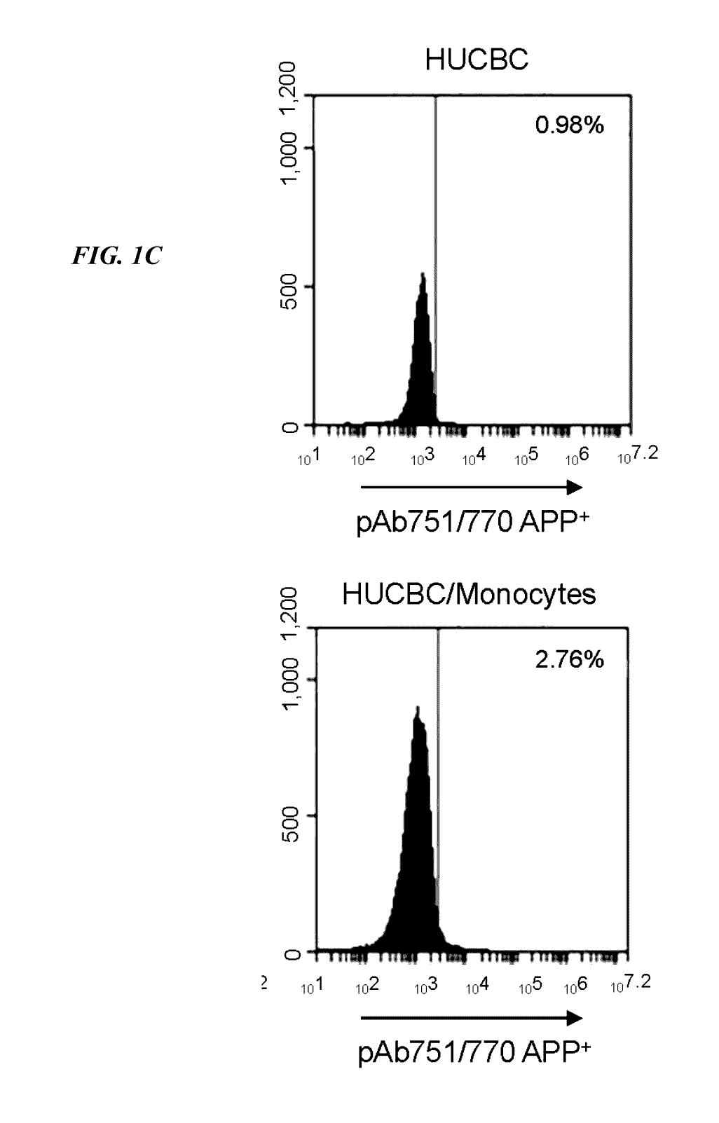

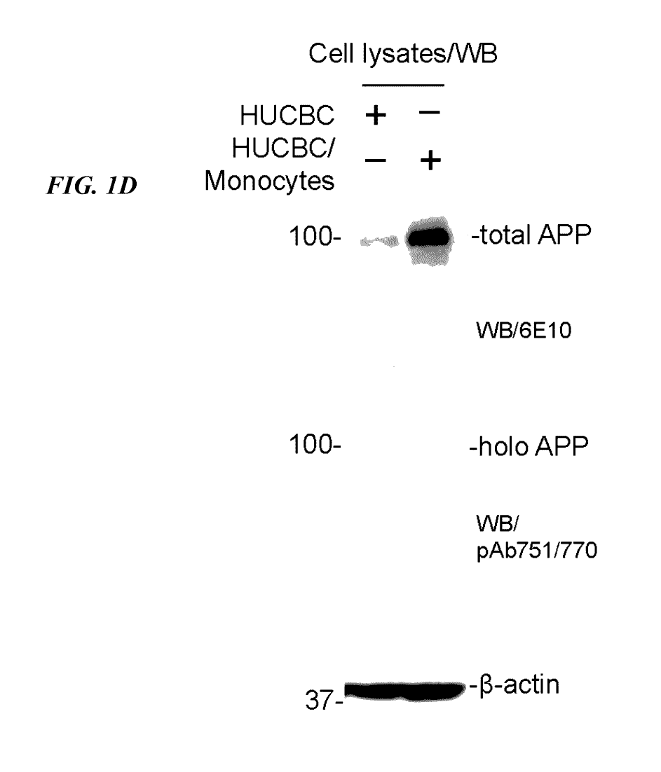

CD14⁺ monocytes were isolated from HUCBCs using a MACS Miltenyi biotec CD14⁺ MicroBeads human kit via positive selection and then characterized by FACS analysis. Prior to enrichment, there was a 13% CD14⁺ monocyte population (FIG. 1A, upper panel), whereas after positive selection, the population increased to 95% (FIG. 1A, lower panel). HUCBC-derived monocytes were further investigated for surface and full-length APP using 6E10 and pAb751/770 antibodies respectively. Antibody 6E10 recognizes total APP, Aβ, and β-CTF, while pAb751/770 antibody specifically recognizes full-length APP. Unfractionated HUCBCs and purified CD14⁺ monocytes were 48.0% and 93.8% positive for surface APP (FIG. 1B), respectively. Conversely, pAb751/770 antibody detected only 0.98% and 3% full length APP in unfractionated HUCBCs and purified CD14+ monocytes, respectively, indicating full-length APP may have been truncated (FIG. 1C). To further confirm these data, HUCBCs and HUCBC-derived monocyte lysates were analyzed by Western blot (WB) analysis, indicating that HUCBC-derived monocytes express dramatically more total APP than the whole HUCBCs. The pAb751/770 antibody did not detect any band in both whole HUCBCs and HUCBC-derived monocytes fractions (FIG. 1D).

ii. Short Term (Two-Month) Administration of HUCBC-Derived Monocytes Improves Learning, Memory and Motor Function in PSAPP and WT Mice

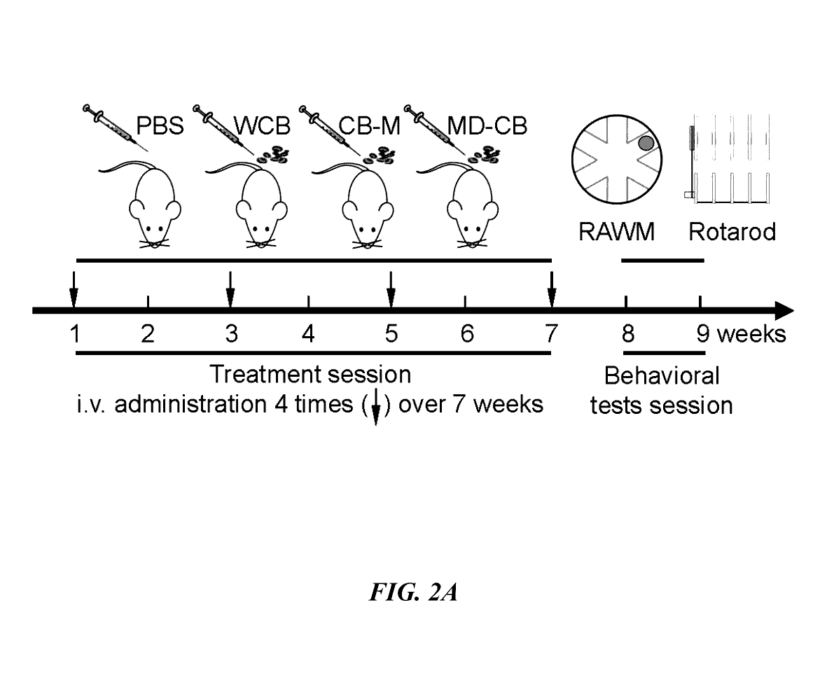

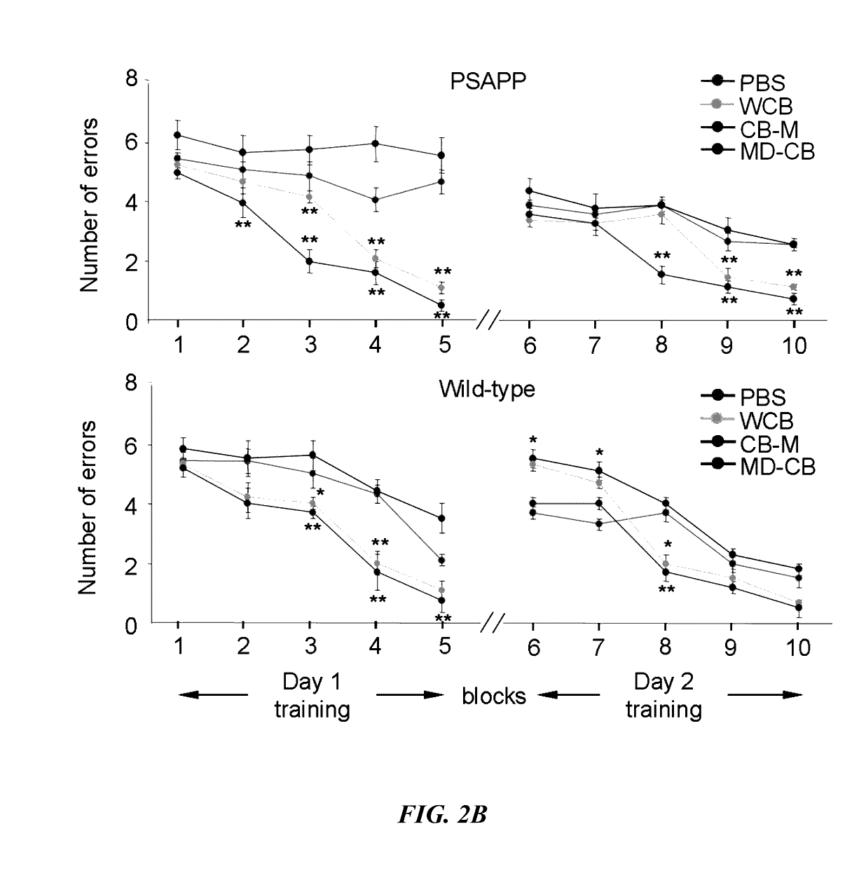

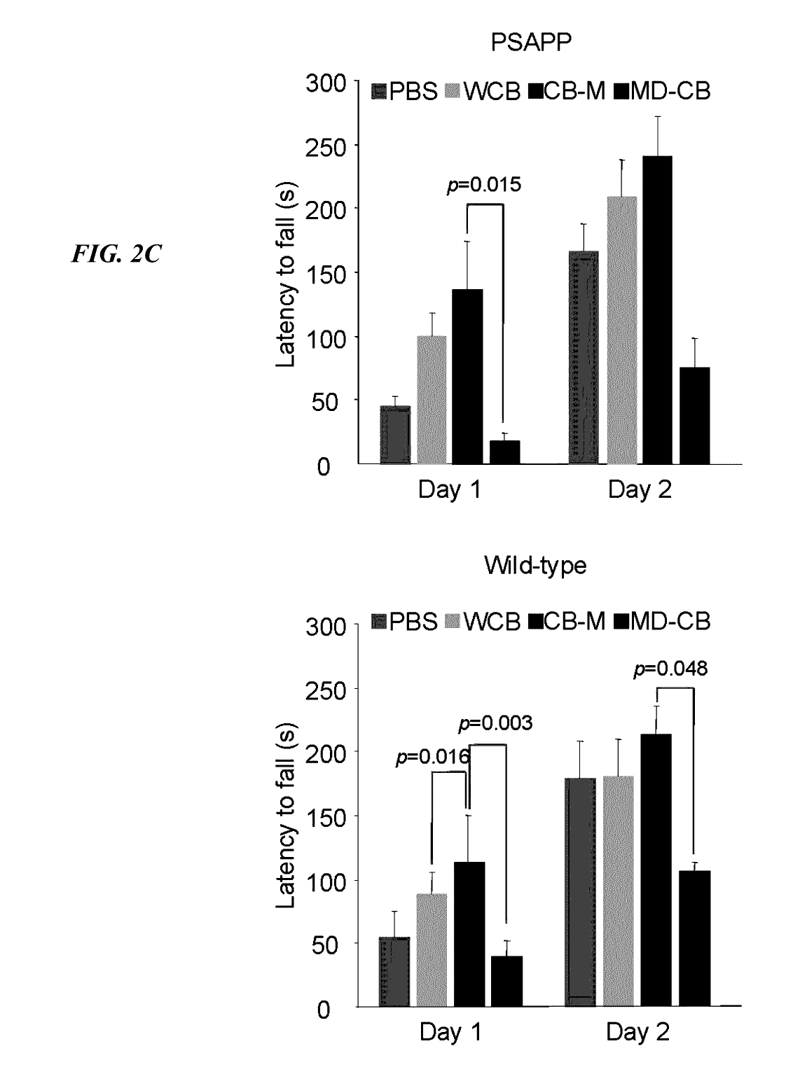

To investigate whether HUCBC-derived monocytes could improve locomotive and cognitive function, PBS, whole HUCBCs (WCB), HUCBC-derived monocytes (CB-M), or monocyte-deficient HUCBC (MD-CB) was administered intravenously in PSAPP and control (aged-matched WT) mice over a two-month time period and then subjected them to rotarod and RAWM testing. (FIG. 2A). Beginning with the block 2 trial, CB-M- and WCB-treated PSAPP mice showed significantly fewer errors in RAWM than PBS and MD-CB-treated PSAPP mice (FIG. 2B). **P<0.01 versus PBS-PSAPP mice). Likewise, WCB- and CB-M-treated WT mice located the target with significantly fewer errors than PBS-treated WT mice during blocks 3 to 5 and 8 (*P<0.05, **P<0.01 versus PBS-WT and MD-CB-WT littermates). Interestingly, MD-CB-treated PSAPP mice were cognitively inflexible and MD-CB treated WT mice learned less at block 6 and 7 when compared to their PBS-treated WT littermates (FIG. 2B). FIG. 2C shows significantly higher coordination and balance in CB-M compared to PBS, WCB, or MD-CB treated PSAPP and WT mice. Notably, increased latency to fall over time appeared standard for all mice, while CB-M-treated PSAPP and WT mice displayed overall greater latency to fall. This signified that CB-M-treated PSAPP and WT mice developed enhanced coordination and balance abilities over PBS-treated PSAPP and WT mice.

iii. Long Term (Four-Month) Administration of HUCBC-Derived Monocytes Improves Learning, Memory, and Motor Function in PSAPP and WT Mice

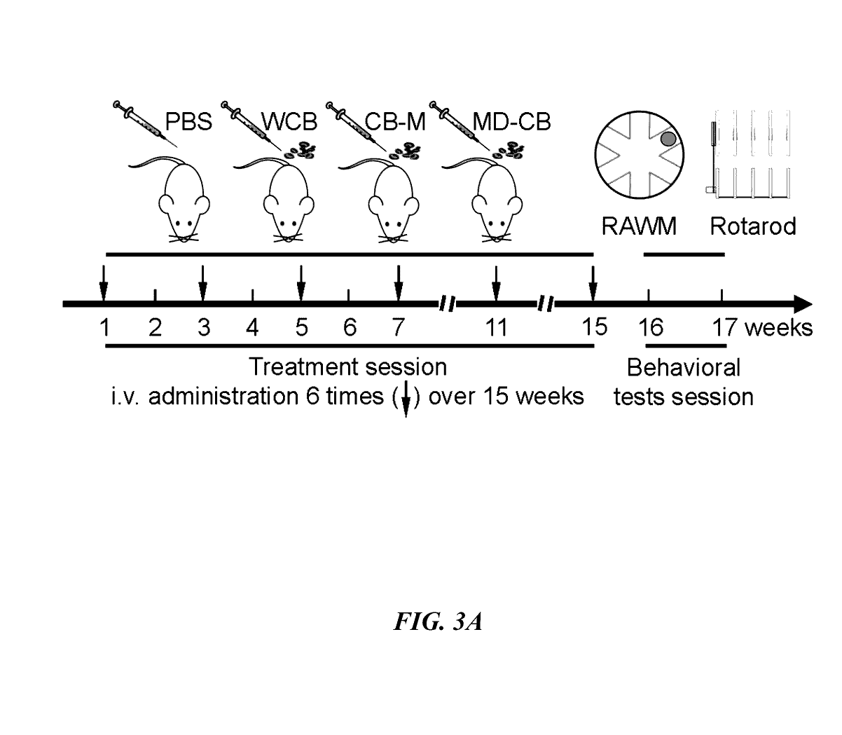

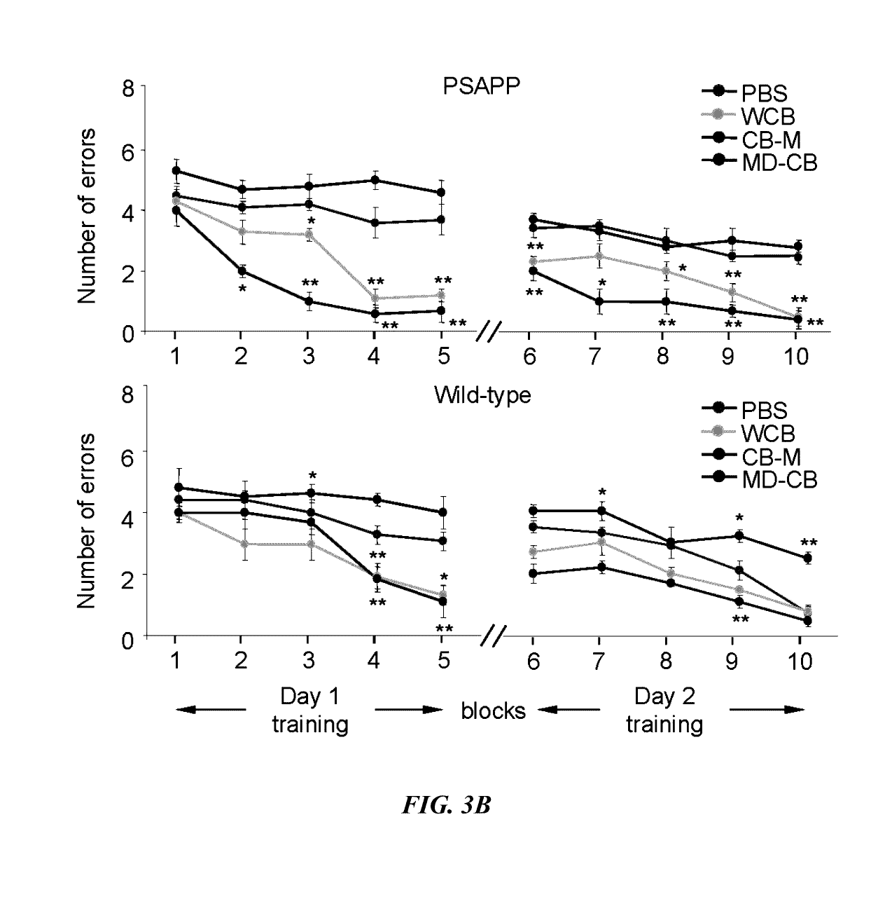

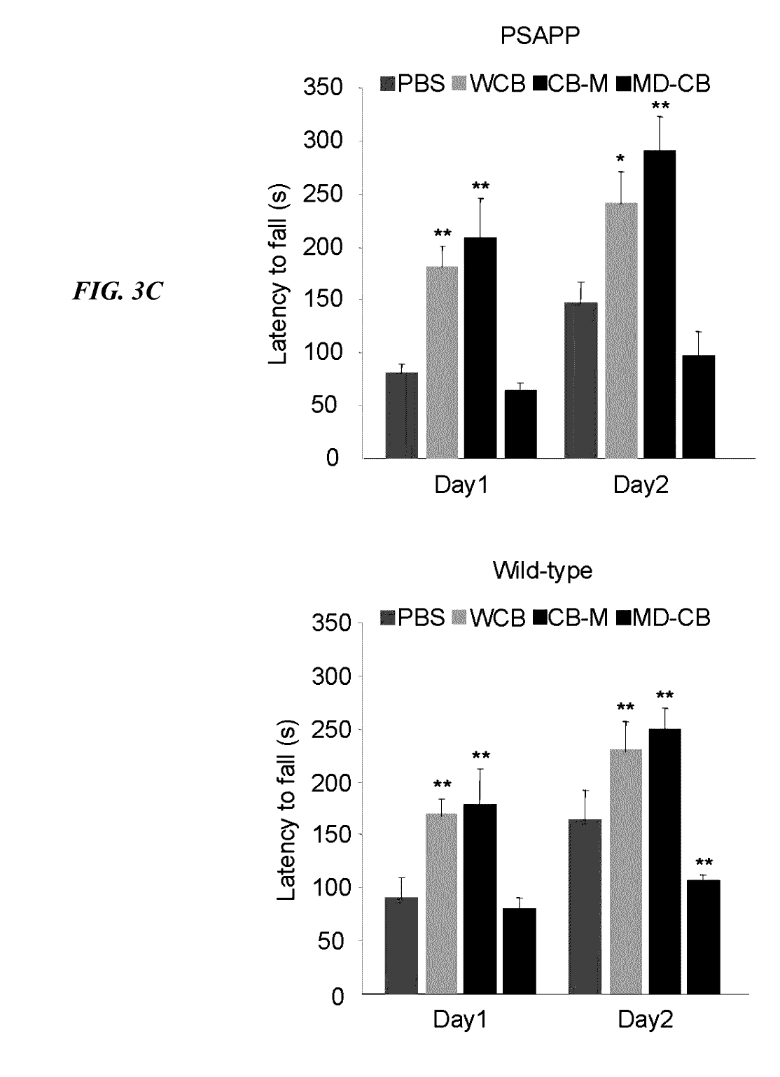

To investigate whether long term (four-month) monocyte administration improves learning, memory, and motor functions, PBS, whole HUCBCs (WCB), HUCBC-derived monocytes (CB-M), or monocyte-deficient HUCBC (MD-CB) was administered intravenously in PSAPP and control (aged-matched WT) mice over a four-month time period. The behavioral data at 4-month of each treatment were reminiscent of the two-month treatment results (FIGS. 3A-3C). In the RAWM, CB-M- and WCB-treated PSAPP mice showed significantly fewer errors (blocks 2 to 5 on day 1; blocks 6 to 10 on day 2; *P<0.05 and **P<0.01) than all other groups. CB-M and WCB-treated WT mice likewise showed significantly fewer errors in blocks 4, 5 and 9, compared to other groups. Surprisingly, PBS-treated WT mice showed better performance than the MD-CB-treated group in blocks 3, 7, 9, and 10 (FIG. 3B). These findings were further reinforced using the open pool platform swim test to assess whether animals possessed the motor skills sufficient to complete the water maze tasks. Data indicate both two and four-month CB-M-treatment outperformed PBS-treatment of PSAPP and WT mice at short and long term treatment periods (data not shown).

Rotarod data show that WCB- and CB-M-treated PSAPP and WT mice displayed superior coordination and advanced to the point of sustaining their position on the rod for the entire trial time of 5 minutes (FIG. 3C; day 2), compared to both PBS-treated PSAPP and WT groups (*P<0.05, **P<0.01 versus PBS-PSAPP or WT mice).

iv. HUCBC-Derived Monocytes Markedly Reduce Aβ Deposits

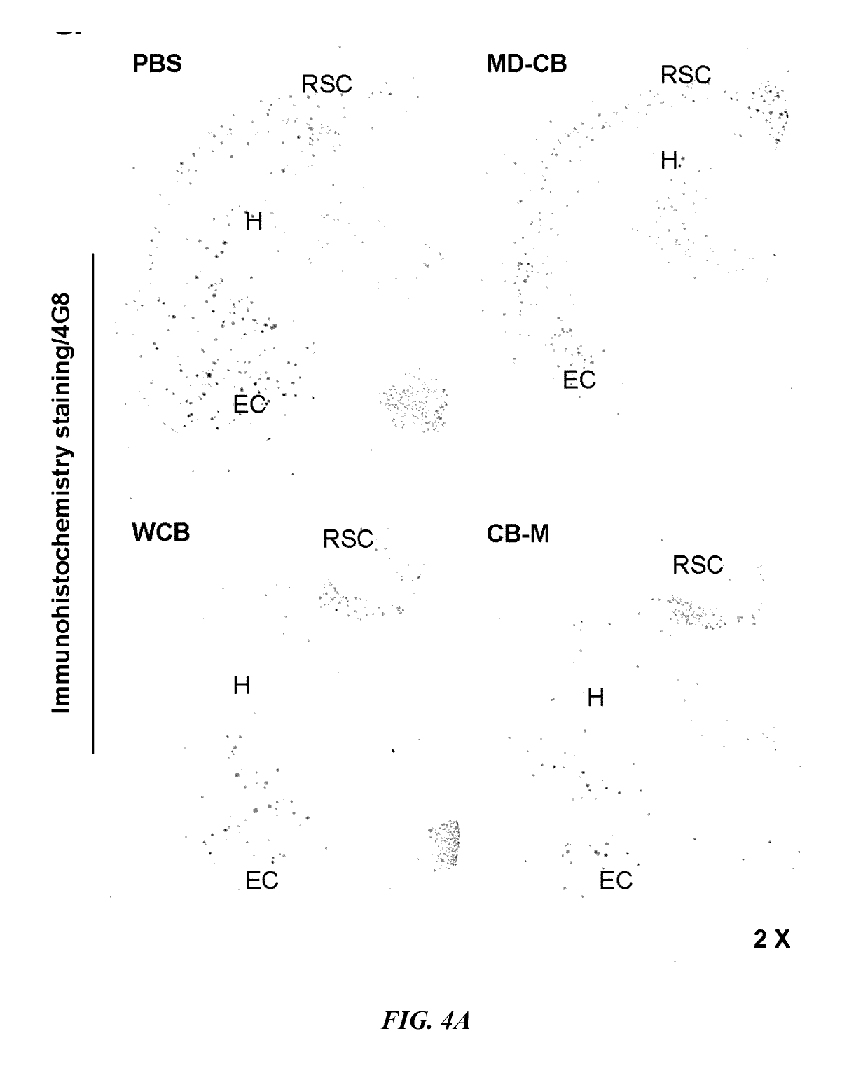

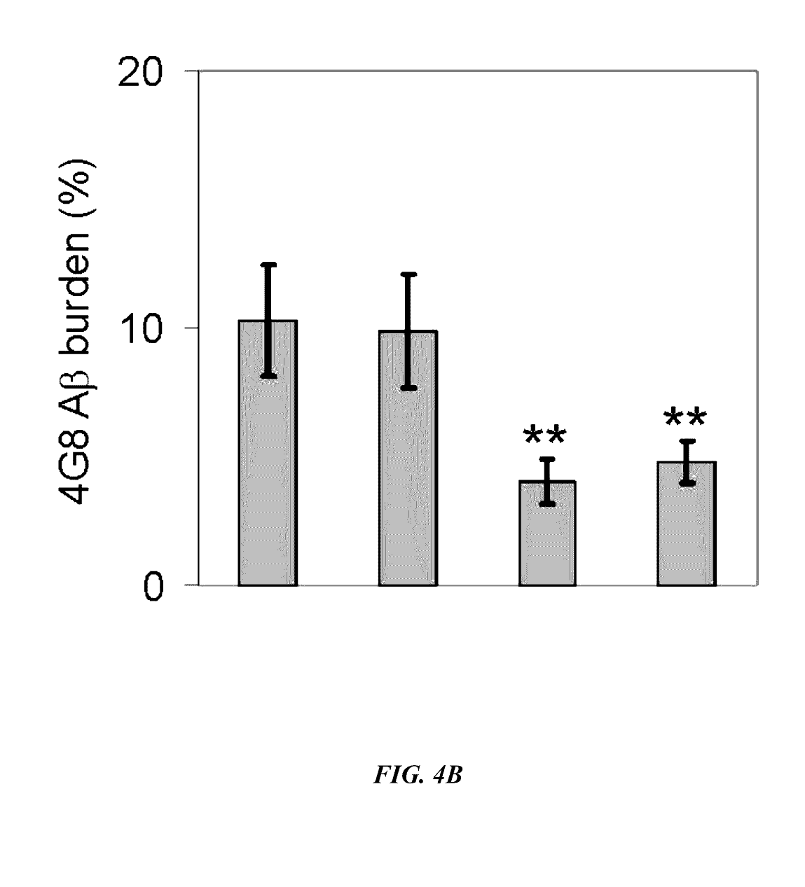

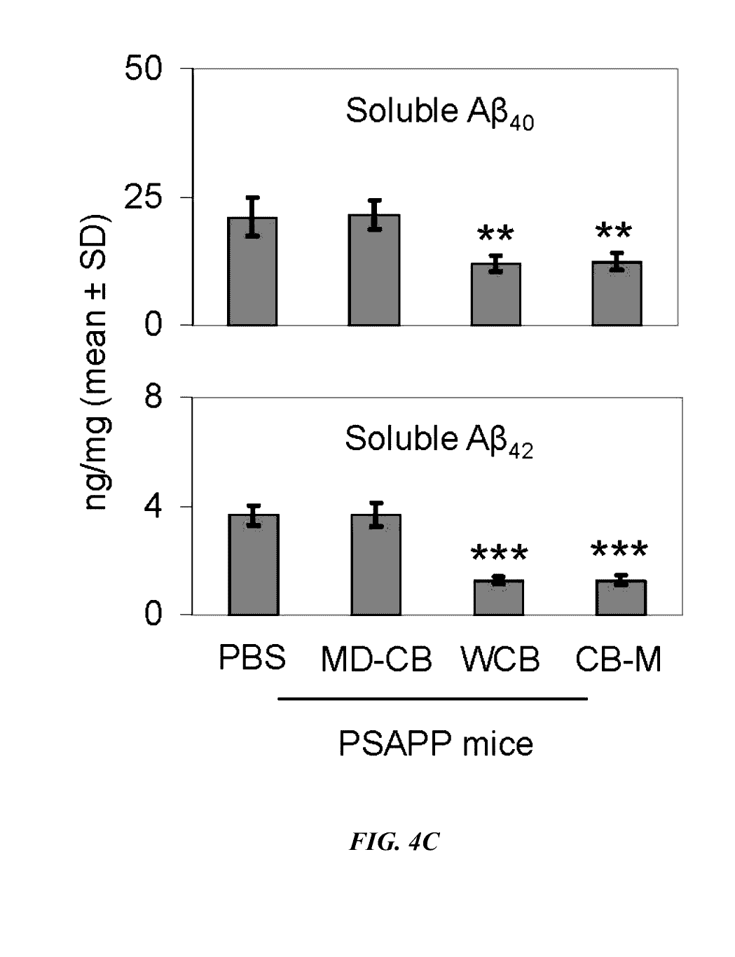

To investigate whether HUCBC-derived monocytes could reduce amyloid pathology, the brains of each mouse were investigated after four different treatments (i.e., PBS, whole HUCBCs (WCB), HUCBC-derived monocytes (CB-M), or monocyte-deficient HUCBC (MD-CB) treatments). WCB- and CB-M-treated PSAPP mice showed reduced amyloid plaques in hippocampus (H), retrosplenial cortex (RSC), and entorhinal (EC) regions as compared with PBS- and MD-CB-treated mice using 4G8 immunohistochemical staining (FIG. 4A). Quantitative image analysis shows that percentage of plaques in the H region in CB-M and WCB treated groups was significantly less than PBS- and MD-CB groups (FIG. 4B, upper panel, mean±SD, n=4).

In addition, brain homogenates from the four-month treatment groups were measured for both soluble Aβ₁₋₄₀ and Aβ₁₋₄₂ levels by ELISA. WCB- and CB-M-treated PSAPP mice showed significantly decreased levels of both soluble Aβ₁₋₄₀ and Aβ₁₋₄₂ compared to PBS and MD-CB-treated group (FIG. 4C, middle and lower panels, mean±SD, n=4). The two-month treatment resulted in similar outcomes (data not shown).

v. Aβ Phagocytosis by Aged and Cord Blood Cell-Derived Monocytes

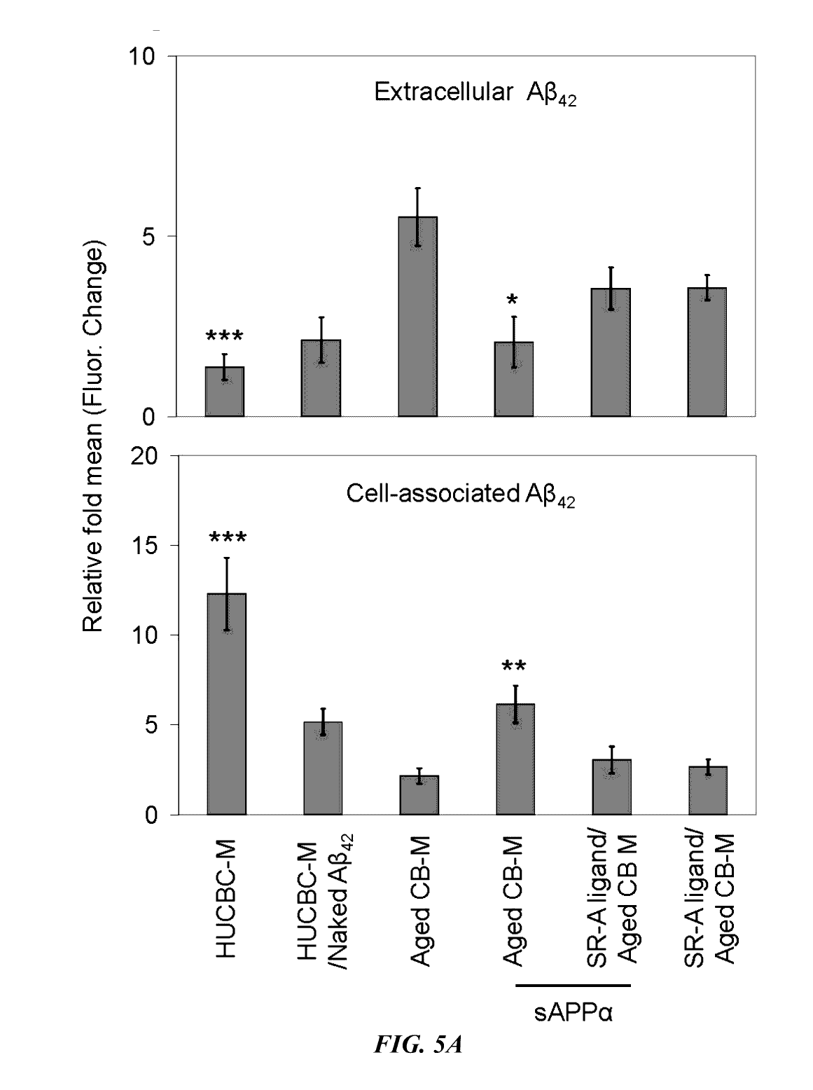

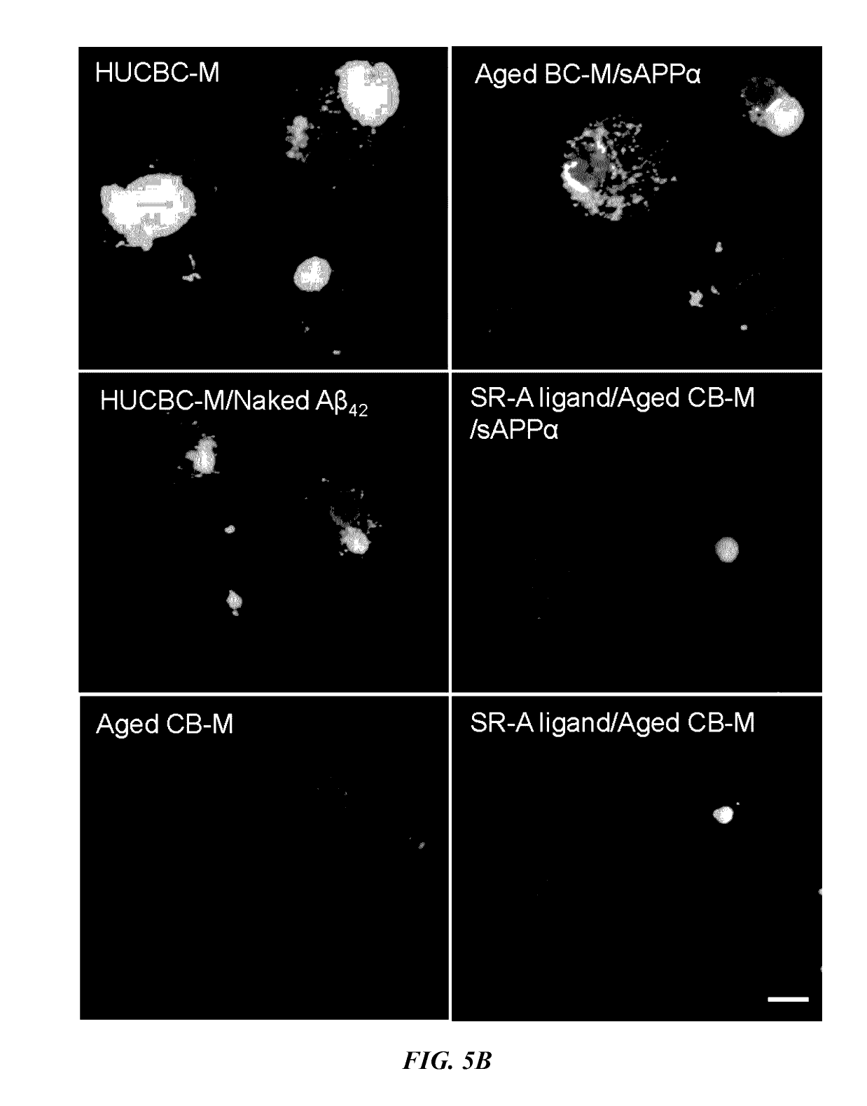

Since phagocytosis by monocytes may be a possible pathway for Aβ clearance from the brain (31,14), it was hypothesized that HUCBC-derived monocytes might phagocytose Aβ HUCBC-derived monocytes (CB-M) and aged human blood cell-derived monocytes (Aged BC-M) were cultured with FITC-Aβ₁₋₄₂ for 1 h in the presence or absence of sAPPα (100 ng/mL), scavenger receptor class A ligand (SR-A ligand) or unlabeled Aβ₁₋₄₂ (Naked Aβ₁₋₄₂). Extracellular (FIG. 5A, upper panel) and cell-associated (lower panel) FITC-Aβ₁₋₄₂ was then measured from supernatants and lysates respectively using a fluorimeter.

Data are represented as relative mean fluorescence (mean±SD) for each sample at 37° C. divided by mean fluorescence at 4° C. (n=4 for each condition) (*P<0.05, **P<0.01, ***P<0.001 versus aged BC-M). Aβ internalization was reflected by the level of cell-associated FITC-A Aβ₁₋₄₂. Interestingly, CB-M internalized Aβ much more than aged CB-M, while sAPPα pretreatment (100 ng/ml) enhanced Aβ internalization by aged BC-M. SR-A ligand reduced Aβ internalization by aged CB-M during sAPPα treatment, suggesting that SR-A is a primary cell surface receptor accounting for Aβ internalization by these cells. Naked Aβ₁₋₄₂ reduced Aβ internalization by HUCBC-M by competitive inhibition.

Primary monocytes were then fixed and imaged using confocal microscopy equipped with Normarski optics (FIG. 5B). Merged image showed significant Aβ₄₂ internalization inside the cytoplasm of CB-M as shown by most of the green fluorescence localized inside the cytoplasmic compartment (left upper panel) while aged CB-M showed much less FITC-Aβ₁₋₄₂ internalization (left lower panel). Treatment of aged CB-M with sAPPα showed significant increase of Aβ₁₋₄₂ internalization in the cytoplasm by these cells (right upper panel) while SR-A ligand reduced this effect (right middle panel). As expected, competition by naked Aβ₁₋₄₂ significantly decreased FITC-Aβ₁₋₄₂ internalization by CB-M (left middle panel).

vi. In Vitro Heterodimerization sAPPα with Aβ

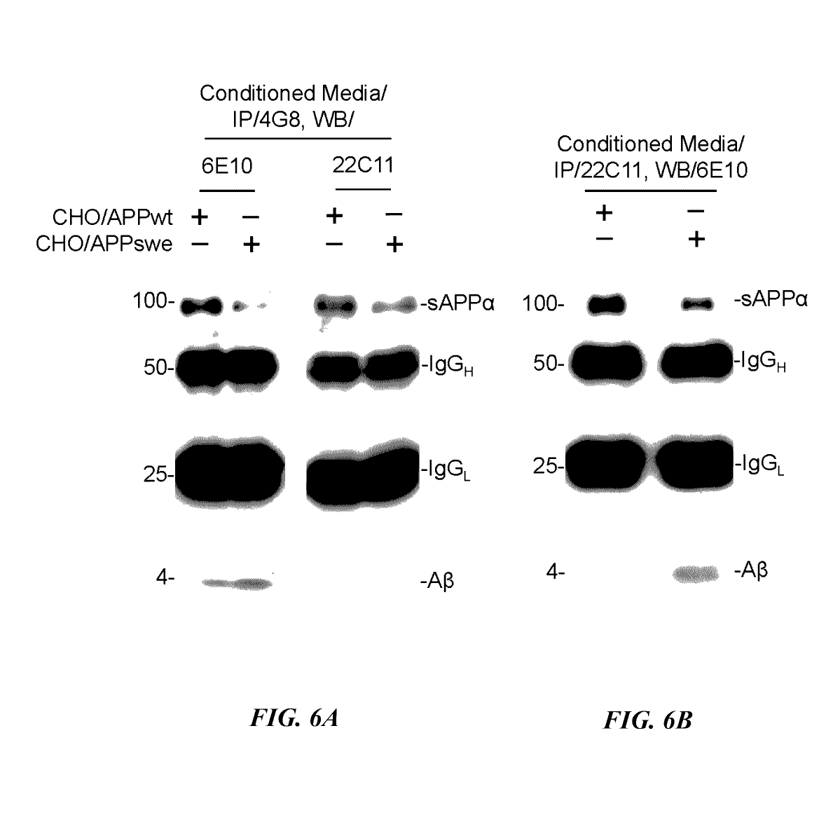

In this study, it was shown that sAPPα treatment improves Aβ phagocytosis by aged blood monocytes. It is known that APP undergoes homodimerization at the cell surface, while sAPPα disrupts this homodimerization by binding with APP, thereby preventing starvation-induced cell death (18)]. It was hypothesized that sAPPα might also form a heterodimer with Aβ, which is then internalized inside the monocytes. To prove sAPPα/Aβ heterodimerization, 3-hour cultured conditioned media of CHO/APPwt or CHO/APPswe cells were immunoprecipitated with anti-Aβ₁₇₋₂₇ antibody (4G8), followed by WB detection using either anti-Aβ₁₋₁₆ antibody (6E10) (FIG. 6A, left panel) or anti-APP N-terminal antibody (22C11) (FIG. 6A, right panel). The 22C11 blot showed only the sAPPα band (˜100 kDa), while the 6E10 blot showed both Aβ (˜4 kDa) and sAPPα (˜100 kDa). Moreover, immunoprecipitation with 22C11, followed by WB detection using 6E10 showed both Aβ (˜4 kDa) and sAPPα (˜100 kDa) bands (FIG. 6B). Taken together, these data suggest a possible sAPPα/Aβ heterodimerization in the cell culture system.

vii. sAPPα/Aβ Heterodimerization at APP₆₇₂₋₆₈₇ (Aβ₁₋₁₆) Region

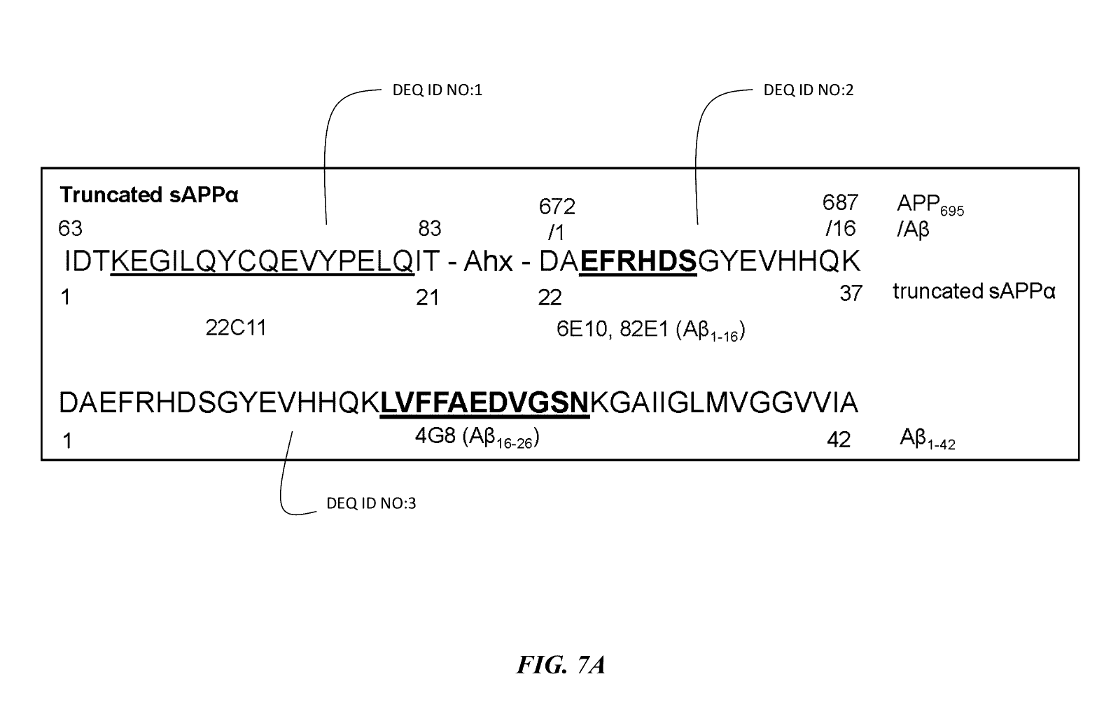

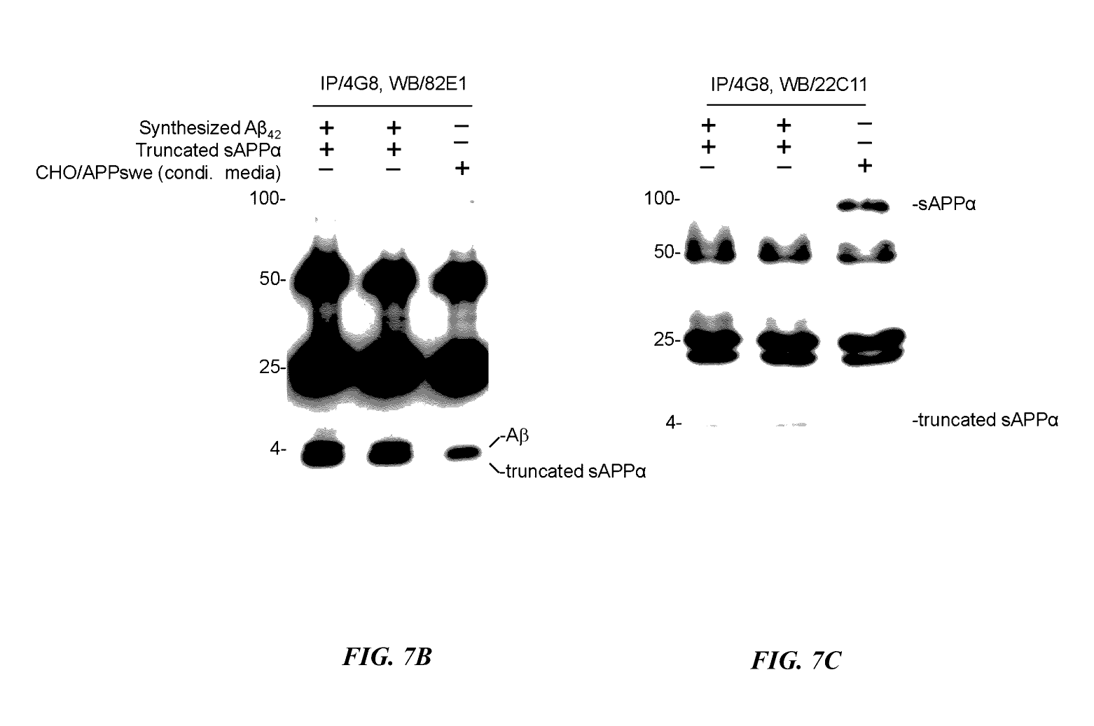

To investigate whether APP₆₇₂₋₆₈₇ region is responsible for sAPPα/Aβ heterodimerization, truncated sAPPα was incubated with synthetic Aβ₁₋₄₂ (FIG. 7A), immunoprecipitated with 4G8, and then analyzed by WB detection using either anti-Aβ₁₋₁₆ antibody (82E1) (FIG. 7B) or anti-APP N-terminal antibody (22C11) (FIG. 7C). In parallel, CHO/APPswe conditioned media received the same treatments as a negative control. Truncated sAPPα incubated with synthetic Aβ₁₋₄₂ showed two attached bands with the presence of not only Aβ₁₋₄₂ (˜4 kDa, upper band) but also truncated sAPPα (˜4 kDa, lower band) (FIG. 7B), while CHO/APPswe conditioned media showed only a single band for Aβ₁₋₄₂ (˜4 kDa). In addition, CHO/APPswe conditioned media clearly show one band for sAPPα (˜100 kDa) while truncated sAPPα incubated with synthetic Aβ₁₋₄₂ clearly shows the presence of truncated sAPPα (˜4 kDa) in the same blot. The presence of sAPPα was substantiated by 6E10 WB (data not shown).

### C. DISCUSSION AND CONCLUSION

The findings of this study suggest that peripheral administration of HUCBC-derived monocytes can improve hippocampal dependent learning, memory, and motor function in transgenic PSAPP mice. Histological and biochemical analyses of brain tissue reveal that both short (two-month) and long term (four-month) administration of HUCBC-monocytes reduces amyloid pathology as well as soluble and insoluble Aβ in the brain.

Confocal microscopy of culture data clearly show that the phagocytic capacity of aged monocytes decreased significantly and was restored by sAPPα treatment (FIGS. 5A-5B), indicating that this protein might have an important therapeutic role in reducing Aβ from the brain parenchyma. Furthermore, the lower phagocytic capacity of aged blood monocytes could be explained by significantly less APP surface expression in aged blood monocytes and therefore correspondingly much lower sAPPα secretion than HUCBC-derived monocytes (shown in FIGS. 5A-5B). It has been shown that the SR-A receptor binds with Aβ and promotes internalization and clearance (22,50,16) and microglial expression of SR-A increase surrounding human brain plaques area (8).

The most intriguing part of this study was to identify one of the plausible mechanisms involved in Aβ phagocytosis by monocytes. Confocal microscopy and immunoprecipitation technique show that sAPPα binds with Aβ₄₂ species at the cell surface which promote internalization of Aβ peptide inside the monocyte's cytoplasm. It was hypothesized that heterodimerization of sAPPα/Aβ could be a plausible mechanism by which monocytes or peripheral macrophages reduce amyloid burden from brain parenchyma. The in vitro experiment showed that the APP₆₇₂₋₆₈₇ (Aβ₁₋₁₆) region is responsible for sAPPα/Aβ heterodimerization. Many researchers have emphasized that size of Aβ aggregates is crucial for internalization. Weltzien et al. found that smaller aggregates internalized efficiently but larger aggregates usually spend more time on the monocyte's cell surface (45). Thus, the physical limit of Aβ aggregated mass size should be considered for phagocytosis and inflammation aggravation. In sum, it was found that HUCBC-monocytes can be a novel therapeutic option in AD.

### D. REFERENCES

1. Arendash G W, King D L, Gordon M N, Morgan D, Hatcher J M, et al. (2001) Progressive, age-related behavioral impairments in transgenic mice carrying both mutant amyloid precursor protein and presenilin-1 transgenes. Brain Res 891: 42-53.
2. Bandyopadhyay S, Goldstein L E, Lahiri D K, Rogers J T (2007) Role of the APP non-amyloidogenic signaling pathway and targeting alpha-secretase as an alternative drug target for treatment of Alzheimer's disease. Curr Med Chem. 14: 2848-2864.
3. Bao Y, Kim E, Bhosle S, Mehta H, Cho S (2010) A role for spleen monocytes in post-ischemic brain inflammation and injury. J Neuroinflammation 7: 92.
4. Barten D M, Albright C F (2008) Therapeutic strategies for Alzheimer's disease. Mol Neurobiol 37: 171-186.
5. Borchelt D R, Ratovitski T, van Lare J, Lee M K, Gonzales V, et al. (1997) Accelerated amyloid deposition in the brains of transgenic mice coexpressing mutant presenilin 1 and amyloid precursor proteins. Neuron 19: 939-945.
6. Brunstein C G (2011) Umbilical cord blood transplantation for the treatment of hematologic malignancies. Cancer Control 18: 222-236.
7. Chen J, Sanberg P R, Li Y, Wang L, Lu M, et al. (2001) Intravenous administration of human umbilical cord blood reduces behavioral deficits after stroke in rats. Stroke 32: 2682-2688.
8. Christie R H, Freeman M, Hyman B T (1996) Expression of the macrophage scavenger receptor, a multifunctional lipoprotein receptor, in microglia associated with senile plaques in Alzheimer's disease. Am J Pathol 148: 399-403.
9. Darlington D, Deng J, Giunta B, Hou H, Sanberg C D, et al. (2013) Multiple low-dose infusions of human umbilical cord blood cells improve cognitive impairments and reduce amyloid-beta-associated neuropathology in Alzheimer mice. Stem Cells Dev 22: 412-421.
10. Deane R, Wu Z, Sagare A, Davis J, Du Yan S, et al. (2004) LRP/amyloid beta-peptide interaction mediates differential brain efflux of Abeta isoforms. Neuron. 43: 333-344.
11. Dewachter I, Van Leuven F (2002) Secretases as targets for the treatment of Alzheimer's disease: the prospects. Lancet Neurol. 1: 409-416.
12. D'Mello C, Le T, Swain M G (2009) Cerebral microglia recruit monocytes into the brain in response to tumor necrosis factoralpha signaling during peripheral organ inflammation. J Neurosci 29: 2089-2102.
13. Donnelly D J, Popovich P G (2008) Inflammation and its role in neuroprotection, axonal regeneration and functional recovery after spinal cord injury. Exp Neurol 209: 378-388.
14. Fiala M, Lin J, Ringman J, Kermani-Arab V, Tsao G, et al. (2005) Ineffective phagocytosis of amyloid-beta by macrophages of Alzheimer's disease patients. J Alzheimers Dis 7: 221-232; discussion 255-262.
15. Floris S, Blezer E L, Schreibelt G, Dopp E, van der Pol S M, et al. (2004) Blood-brain barrier permeability and monocyte infiltration in experimental allergic encephalomyelitis: a quantitative Mill study. Brain 127: 616-627.
16. Frenkel D, Wilkinson K, Zhao L, Hickman S E, Means T K, et al. (2013) Scaral deficiency impairs clearance of soluble amyloid-beta by mononuclear phagocytes and accelerates Alzheimer's-like disease progression. Nat Commun 4: 2030.
17. Garcia-Alloza M, Robbins E M, Zhang-Nunes S X, Purcell S M, Betensky R A, et al. (2006) Characterization of amyloid deposition in the APPswe/PS1DE9 mouse model of Alzheimer disease. Neurobiol Dis 24: 516-524.
18. Gralle M, Botelho M G, Wouters F S (2009) Neuroprotective secreted amyloid precursor protein acts by disrupting amyloid precursor protein dimers. J Biol Chem 284: 15016-15025.
19. Hardy J (2006) Has the amyloid cascade hypothesis for Alzheimer's disease been proved? Curr Alzheimer Res 3: 71-73.
20. Hawkes C A, McLaurin J (2009) Selective targeting of perivascular macrophages for clearance of beta-amyloid in cerebral amyloid angiopathy. Proc Natl Acad Sci USA 106: 1261-1266.
21. Henning R J, Burgos J D, Ondrovic L, Sanberg P, Balis J, et al. (2006) Human umbilical cord blood progenitor cells are attracted to infarcted myocardium and significantly reduce myocardial infarction size. Cell Transplant 15: 647-658.
22. Husemann J, Loike J D, Anankov R, Febbraio M, Silverstein S C (2002) Scavenger receptors in neurobiology and neuropathology: their role on microglia and other cells of the nervous system. Glia 40: 195-205.
23. Jankowsky J L, Slunt H H, Gonzales V, Jenkins N A, Copeland N G, et al. (2004) APP processing and amyloid deposition in mice haplo-insufficient for presenilin 1. Neurobiol Aging 25: 885-892.
24. Kang D E, Pietrzik C U, Baum L, Chevallier N, Merriam D E, et al. (2000) Modulation of amyloid beta-protein clearance and Alzheimer's disease susceptibility by the LDL receptor-related protein pathway. J Clin Invest. 106: 1159-1166.
25. Kigerl K A, Gensel J C, Ankeny D P, Alexander J K, Donnelly D J, et al. (2009) Identification of two distinct macrophage subsets with divergent effects causing either neurotoxicity or regeneration in the injured mouse spinal cord. J Neurosci 29: 13435-13444.
26. Kim E, Yang J, Beltran C D, Cho S (2014) Role of spleen-derived monocytes/macrophages in acute ischemic brain injury. J Cereb Blood Flow Metab 34: 1411-1419.
27. Lemere, C.; Masliah, E.; (2010) Can Alzheimer disease be prevented by amyloid-beta immunotherapy? Nat Rev Neurol 6: 108-119.
28. Leor J, Guetta E, Chouraqui P, Guetta V, Nagler A (2005) Human umbilical cord blood cells: a new alternative for myocardial repair? Cytotherapy 7: 251-257.
29. Lu D, Sanberg P R, Mahmood A, Li Y, Wang L, et al. (2002) Intravenous administration of human umbilical cord blood reduces neurological deficit in the rat after traumatic brain injury. Cell Transplant 11: 275-281.
30. McGavern D B, Kang S S (2011) Illuminating viral infections in the nervous system. Nat
Rev Immunol 11: 318-329.
31. Michaud J P, Bellavance M A, Prefontaine P, Rivest S (2013) Real-time in vivo imaging reveals the ability of monocytes to clear vascular amyloid beta. Cell Rep 5: 646-653.
32. Mildner A, Schlevogt B, Kierdorf K, Bottcher C, Erny D, et al. (2011) Distinct and non-redundant roles of microglia and myeloid subsets in mouse models of Alzheimer's disease. J Neurosci 31: 11159-11171.
33. Miyake T, Tsuchihashi Y, Kitamura T, Fujita S (1984) Immunohistochemical studies of blood monocytes infiltrating into the neonatal rat brain. Acta Neuropathol 62: 291-297.
34. Murphy M P, Beckett T L, Ding Q, Patel E, Markesbery W R, et al. (2007) Abeta solubility and deposition during AD progression and in APPxPS-1 knock-in mice. Neurobiol Dis 27: 301-311.
35. Nazer B, Hong S, Selkoe D J (2008) LRP promotes endocytosis and degradation, but not transcytosis, of the amyloid-beta peptide in a blood-brain barrier in vitro model. Neurobiol Dis. 30: 94-102.
36. Nikolic W V, Hou H, Town T, Zhu Y, Giunta B, et al. (2008) Peripherally administered human umbilical cord blood cells reduce parenchymal and vascular beta-amyloid deposits in Alzheimer mice. Stem Cells Dev 17: 423-439.
37. Rezai-Zadeh K, Shytle D, Sun N, Mori T, Hou H, et al. (2005) Green tea epigallocatechin-3-gallate (EGCG) modulates amyloid precursor protein cleavage and reduces cerebral amyloidosis in Alzheimer transgenic mice. J Neurosci 25: 8807-8814.
38. Saporta S, Kim J J, Willing A E, Fu E S, Davis C D, et al. (2003) Human umbilical cord blood stem cells infusion in spinal cord injury: engraftment and beneficial influence on behavior. J Hematother Stem Cell Res 12: 271-278.
39. Selkoe D J (1998) The cell biology of beta-amyloid precursor protein and presenilin in Alzheimer's disease. Trends Cell Biol. 8: 447-453.
40. Seta N, Kuwana M (2007) Human circulating monocytes as multipotential progenitors. Keio J Med 56: 41-47.
41. Shibata M, Yamada S, Kumar S R, Calero M, Bading J, et al. (2000) Clearance of Alzheimer's amyloid-ss (1-40) peptide from brain by LDL receptor-related protein-1 at the blood-brain barrier. J Clin Invest. 106: 1489-1499.
42. Tan J, Town T, Crawford F, Mori T, DelleDonne A, et al. (2002) Role of CD40 ligand in amyloidosis in transgenic Alzheimer's mice. Nat Neurosci 5: 1288-1293.
43. Van Uden E, Mallory M, Veinbergs I, Alford M, Rockenstein E, et al. (2002) Increased extracellular amyloid deposition and neurodegeneration in human amyloid precursor protein transgenic mice deficient in receptor-associated protein. J Neurosci. 22: 9298-9304.
44. Vitale S, Schmid-Alliana A, Breuil V, Pomeranz M, Millet M A, et al. (2004) Soluble fractalkine prevents monocyte chemoattractant protein-1-induced monocyte migration via inhibition of stress-activated protein kinase 2/p38 and matrix metalloproteinase activities. J Immunol 172: 585-592.
45. Weltzien R B, Pachter J S (2000) Visualization of beta-amyloid peptide (Abeta) phagocytosis by human mononuclear phagocytes: dependency on Abeta aggregate size. J Neurosci Res 59: 522-527.
46. Willing A E, Lixian J, Milliken M, Poulos S, Zigova T, et al. (2003) Intravenous versus intrastriatal cord blood administration in a rodent model of stroke. J Neurosci Res 73: 296-307.
47. Wohleb E S, Hanke M L, Corona A W, Powell N D, Stiner L M, et al. (2011) beta-Adrenergic receptor antagonism prevents anxiety-like behavior and microglial reactivity induced by repeated social defeat. J Neurosci 31: 6277-6288.
48. Wohleb E S, McKim D B, Shea D T, Powell N D, Tarr A J, et al. (2014) Re-establishment of anxiety in stress-sensitized mice is caused by monocyte trafficking from the spleen to the brain. Biol Psychiatry 75: 970-981.
49. Wohleb E S, Powell N D, Godbout J P, Sheridan J F (2013) Stress-induced recruitment of bone marrow-derived monocytes to the brain promotes anxiety-like behavior. J Neurosci 33: 13820-13833.
50. Yang C N, Shiao Y J, Shie F S, Guo B S, Chen P H, et al. (2011) Mechanism mediating oligomeric Abeta clearance by naive primary microglia. Neurobiol Dis 42: 221-230.
51. Yazawa H, Yu Z X, Takeda, Le Y, Gong W, et al. (2001) Beta amyloid peptide (Abeta42) is internalized via the G-protein-coupled receptor FPRL1 and forms fibrillar aggregates in macrophages. FASEB J 15: 2454-2462.
52. Zhu Y, Bickford P C, Sanberg P, Giunta B, Tan J (2008) Blueberry opposes beta-amyloid peptide-induced microglial activation via inhibition of p44/42 mitogen-activation protein kinase. Rejuvenation Res 11: 891-901.
53. Zhu Y, Hou H, Rezai-Zadeh K, Giunta B, Ruscin A, et al. (2011) CD45 deficiency drives amyloid-beta peptide oligomers and neuronal loss in Alzheimer's disease mice. J Neurosci 31: 1355-1365.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A method of preventing or treating a neurodegenerative disease or disorder, comprising administering a therapeutically effective amount of human umbilical cord blood-derived monocytes.

2. A method as in claim 1, wherein the neurodegenerative disease or disorder is selected from the group consisting of Alzheimer's disease, stroke, and traumatic brain injury.

3. A method as in claim 1, wherein the human umbilical cord blood-derived monocytes are pretreated with exogenous soluble amyloid precursor protein alpha (sAPPα).

4. A method as in claim 2, wherein the human umbilical cord blood-derived monocytes are administered in multiple low-dose infusions.

5. A composition comprising sAPPα-treated aged monocytes formed by heterodimerization of sAPPα with amyloid beta (Aβ).

6. A method of preventing or treating a neurodegenerative disease or disorder, comprising administering a therapeutically effective amount of sAPPα-treated aged monocytes formed by heterodimerization of sAPPα with Aβ.

7. A method as in claim 6, wherein the neurodegenerative disease or disorder is selected from the group consisting of Alzheimer's disease, stroke, and traumatic brain injury.
